(19) United States
(12) Patent Application Publication
(10) Pub. No.: US 2008/0038742 A1
(43) Pub. Date: Feb. 14, 2008

(54) Genetic polymorphisms associated with body fat

(76) Inventors: Tom E. Porter, University Park, MD (US); Michael Robert Muchow, Hyattsville, MD (US)

Correspondence Address: JACOBSON HOLMAN PLLC, 400 SEVENTH STREET N.W., SUITE 600, WASHINGTON, DC, 20004, US

(21) Appl. No.: 11/822,540

(22) Filed: Jul. 6, 2007

Related U.S. Application Data
(60) Provisional application No. 60/807,396, filed on Jul. 14, 2006.

(51) Int. Cl. C12Q 1/68; C07H 21/00

(52) U.S. Cl. 435/6; 536/23.1

(57) ABSTRACT

In the present invention, cDNA microarrays to investigate pituitary gene expression in two chicken lines that were selected for low and high body fat (Lean and Fat). Differentially expressed genes between lines are potential candidates as genetic markers for high and low potential for body fat accumulation. The lysophosphatidic acid (LPA) receptor-1 (LPAR1) was identified as a potential marker, being differentially expressed between the lean and fat lines at the early ages. The invention provides SNPs that can introduce a GATA site in the promoter of LPAR1 which can upregulate its expression in the lean chickens, and increased LPA signaling and which can inhibit preadipocyte differentiation. Conversely, SNPs are provided that cause loss of the GATA binding site and cause decreased levels of LPAR1 expression and attenuated inhibition of adipocyte maturation in fat chickens.

This is a complete application claiming benefit of provisional 60/807,396 filed Jul. 14, 2006 and which are hereby incorporated by reference.

### BACKGROUND OF THE INVENTION

1. Field of Invention

The present invention relates to the identification of genes and single nucleotide polymorphisms (SNPs), which may regulate body fat composition in vertebrates. The present invention also relates to the use of these SNPs to identify and select for lean or fat traits in such animals.

2. Description of Prior Art

The inventors have investigated ways to identify genes expressed in neuroendocrine tissues that regulate body fat composition in domestic chickens using cDNA microarrays. Specifically, the inventors focused on the identification of differentially expressed genes in the anterior pituitary gland of fat and lean chicken lines and analysis of candidate genes to be used for marker-assisted selection of leaner chickens in the future.

Commercial broiler chickens have been selected for increased body mass (i.e. muscle) and rapid growth rate (Havenstein, Ferket et al. 2003). Unfortunately, this selection has also increased body fat deposition (Deeb and Lamont 2002), along with other undesirable traits like decreased reproductive performance, increased skeletal muscle abnormalities, ascites, and fatty liver and kidney syndrome (Griffin and Goddard 1994; Julian 2005). Excessive fat deposition is an undesirable from a production and consumer standpoint, but traditionally commercial selection against this highly heritable trait has not been practiced, due to the cost and labor involved in slaughtering and dissecting animals in breeding assays (Lagarrigue, Pitel et al. 2006). A means of genetically selecting for leanness would be of great commercial value.

Obesity has been described as a rising epidemic in the developed world, especially in the United States, where the prevalence of overweight and obese individuals is increasing. Approximately 60% of the population in the United States is considered overweight or obese [body mass index (BMI, body weight in kilograms over the square of height in meters) greater than 25.0 and 30.0, respectively] (Hedley, Ogden et al. 2004). With this increase in the prevalence of obesity, there has been a concomitant increase in the incidence of other diseases, such as diabetes, hypertension, and cardiovascular disease (Muoio and Newgard 2006). It is likely that any genes involved in body composition phenotype in the chicken have human orthologs, and so may play a role in understanding obesity, which is a major health concern in humans, now and will be in the future.

It is well known that body composition is determined by a complex interaction between environmental, hormonal, genetic, behavioral, and nutritional factors. The pituitary produces (at least) three hormones that exert major effects on growth, body composition, and metabolism: growth hormone (GH), pro-opiomelanacortin (POMC), and thyroidstimulating hormone (TSH). These hormones are produced by somatotrophs, corticotrophs, and thyrotrophs, respectively. Genes involved in the expression and regulation of these hormones, especially during the developmental period during which adipocytes undergo differentiation, were of particular interest with regard to understanding neuroendocrine regulation of adiposity.

There are several peptides produced by the hypothalamus that exert trophic effects on the pituitary gland. The chicken GH-releasing hormone (GHRH) gene has been cloned (McRory, Parker et al. 1997). The preprothyrotropin-releasing hormone (TRH) cDNA has been recently cloned (Vandenborne, Roelens et al. 2005); TRH is a three amino acid peptide (pyro-Glu-His-Pro amine) produced in the paraventricular nucleus (PVN) and in the lateral hypothalamus (Geris, D'Hondt et al. 1999; Vandenborne, Roelens et al. 2005) as a 26 kDa prohormone and is processed into active TRH by the actions of prohormone convertase (PC) 1/3 and PC2 (Perello, Friedman et al. 2006). Corticotropin-releasing hormone (CRH) has been cloned in the chicken (Vandenborne, De Groef et al. 2005). Like mammalian CRH, it is a 41 amino acid peptide found in the paraventricular nucleus (Jozsa, Vigh et al. 1984). Growth hormone GH is necessary for normal growth after hatching (King and Scanes 1986). GH is a 22 kDa glycoprotein hormone synthesized by somatotrophs in the caudal lobe of the chicken anterior pituitary. A cDNA for chicken GH has been sequenced (Lamb, Galehouse et al. 1988). GH expression and secretion are stimulated by GH-releasing hormone (GHRH) (Scanes and Harvey 1984), thyrotropin-releasing hormone (TRH) in embryos and young birds (Van As, Careghi et al. 2004), and ghrelin (Baudet and Harvey 2003), and inhibited by somatotropin-release inhibiting factor (SRIF) (Spencer, Harvey et al. 1986).

Two lines of chickens have been genetically selected that exhibit significant differences in body fat accumulation. The lean and fat chicken lines (LL and FL, respectively) were created at the Institute Nationale Reserches Agrinomique (INRA), Nouzilly, France, and the F0-F2 generations were first described in 1980 (Leclercq, Blum et al. 1980). The selection criterion was based on the proportion of body fat in males at 9 and 16 weeks of age. Generation of the fat and lean broiler strains until the end of selection (F7) was as described previously (Leclercq, Blum et al. 1980; Leclercq 1988). These two strains have now been sustained for more than twenty-five years after selection was discontinued and still maintain the difference in abdominal body fat.

Since the creation of the LL and FL, several differences between the two lines have been noted. For example, in vivo fatty acid synthesis is greater in the FL than the LL, and FL livers are heavier (Saadoun and Leclercq 1983). The LL is more susceptible to depressed growth when fed a low protein diet (Leclercq 1983). The FL birds always have lower blood glucose levels than the LL, whether they are fed or fasted; conversely, the LL always have lower levels of triglycerides, especially in a fed state (Leclercq, Hermier et al. 1984). In short, much work has been done with the FL and LL birds. However, the underlying genetic basis for the differences in abdominal fat is not known. A summary of metabolic differences between LL and FL is presented in Table 1 (Leclercq 1988).

DNA microarrays allow the quantification of expression levels for thousands of genes simultaneously. The construction of the cDNA libraries and the production of the Del-Mar 14K Chicken Integrated Systems Microarray have been described in detail (Cogburn, Wang et al. 2003; Cogburn, Wang et al. 2004; Carre, Wang et al. 2006). The Del-Mar 14K Chicken Integrated Systems Microarrays were printed from cDNA libraries created from metabolic tissues (liver and fat), somatic tissues (skeletal muscle and growth plate), reproductive tissues (oviduct, ovaries, and testes), and neuroendocrine tissues (pituitary, hypothalamus, and pineal). These tissues were chosen for their agricultural and biological importance. All of the publicly available (as of Mar. 1, 2003) chicken expressed sequence tags (ESTs; ˜407000) were assembled into 33949 contigs using the CAP3 software program (Huang and Madan 1999).

The ESTs from the tissue specific libraries were incorporated into these contigs. Contigs were then identified by their highest scoring BLASTX and BLASTN returns. The cDNA clones from the libraries were amplified by PCR and printed onto glass slides. The Del-Mar 14K Chicken Integrated Systems Microarray contains 19200 spots and 14053 of these represent unique cDNA. In addition to the cDNAs from the tissue specific libraries, 387 60-mer oligonucleotide probes for specific genes were printed, along with 72 quality control spots. The quality control spots are salmon sperm DNA, which has been included for an estimation of background hybridization, and 8 housekeeping genes: β-tubulin, TEF1α, β-actin, pre mRNA splicing factor, GAPDH, dynactin, Na+/K+ ATPase, and sodium pump 3 (printed in 8 replicate spots each). The composition of the microarray is summarized in Table 2.

The Del-Mar 14K Chicken Integrated Systems Microarray (GEO accession no. GPL1731) contains 14053 unique cDNAs from multiple tissue specific cDNA libraries (Cogburn, Wang et al. 2003; Cogburn, Wang et al. 2004; Carre, Wang et al. 2006). Of the cDNAs contained in the microarray, the neuroendocrine cDNA library contributed 5929 of these cDNAs, and these neuroendocrine cDNAs have previously been used by the inventors to profile gene expression patterns during pituitary development by microarray analysis (Porter and Ellestad 2005; Ellestad, Carre et al. 2006).

In recent years, there has been a dramatic increase in the available tools for chicken genomics, including the completion of the draft of the chicken genome (Hillier, Miller et al. 2004). The chicken genome is about 1 billion base pairs in sequence containing 20000-23000 genes. Most genetic markers are polymorphic sequences of DNA that have a known locus. Examples of markers are known genes (first generation Type I markers) and much shorter polymorphic segments like microsatellites or variable number tandem repeats (second generation Type II markers) (Emara and Kim 2003). The most common genetic marker is the single nucleotide polymorphism (SNP) and they are considered the basis of third generation genetic maps (Wang, Fan et al. 1998). It is estimated that the chicken genome contains 2.8 million SNPs (Wong, Liu et al. 2004). SNPs can be found anywhere in the genome, but SNPs that are located in the promoter regions of differentially expressed genes will be of particular interest due to the fact that they may alter transcriptional machinery binding sites.

The inventors have taken combined functional genomic and bioinformatic approach in the present invention to analyze differential gene expression in the anterior pituitary between the LL and FL chicken lines at 1-, 3-, 5- and 7-weeks of age (the time frame during which adiposity becomes significantly different) and to identify polymorphisms such as SNPs in the flanking regions of differentially expressed genes that could be used as genetic markers for adiposity.

Until now there has not been an identification of specific genetic markers that correlate with percentage of body fat in a animal, such as a chicken, and in other mammals.

### SUMMARY OF THE INVENTION

It is an object of the present invention to identify genetic markers that could be used to genotype vertebrates for adiposity.

It is an object of the present invention to develop the LPAR-1 marker for use in selective breeding of livestock.

It is an object of the present invention to develop the LPAR-1 marker for use in selective breeding of poultry.

It is also an objection the present invention to create a panel of genes known to be involved in regulating adiposity could be developed for easy assay.

It is a further object of the present invention to provide a kit useful for performing PCR to genotype potential breeders for a number of genes that are markers for adiposity and be selected against in breeding stocks.

It is still another object of the present invention to identify a marker for obesity in mammals and humans.

These and other objects of the invention, as well as many of the attendant advantages thereof, will become more readily apparent when reference is made to the following detailed description of the preferred embodiments.

### BRIEF DESCRIPTION OF THE DRAWINGS

FIG. 1 shows the relationship between abdominal fat to live weight ratio (AF/LW) and live weight (LW) of 9-week-old broilers (F₀).

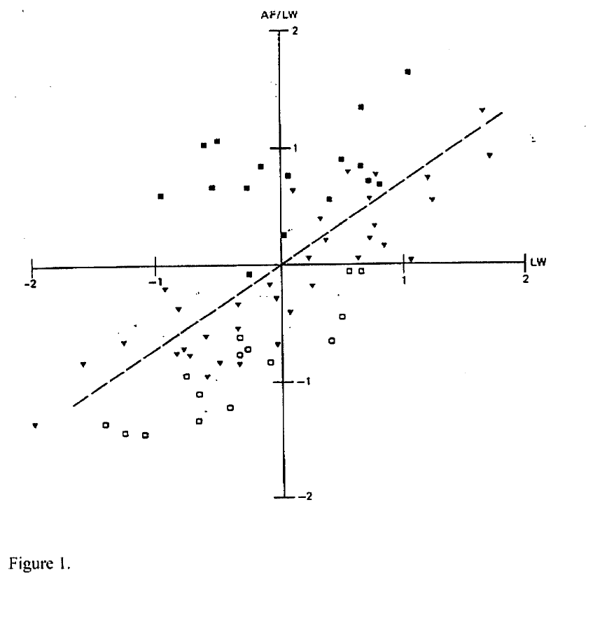

FIG. 2 is a self-organizing map analysis of differentially expressed genes in the Lean and Fat chicken lines.

FIG. 3 is a set of bar graphs showing that gene expression always greater in the Fat line than Lean line.

FIG. 4 is a set of bar graphs showing that gene expression always greater in the Lean line than Fat line.

FIG. 5 shows early age gene expression (weeks 1 and 3) greater in Lean line than Fat line.

FIG. 6 shows late age gene expression (weeks 5 and 7) greater in Lean line than Fat line.

FIG. 7 is another graph showing Early age gene expression greater in Fat line than Lean line.

FIG. 8 is another graph showing late age gene expression greater in Fat line than Lean line.

FIG. 9 is a set of bar graphs illustrating that there were no differences in gene expression between the 2 lines.

FIG. 10 shows gene expression profiles of growth hormone, pro-opiomelanocortin, and thyroid-stimulating hormone beta.

FIG. 11 depicts gene expression profiles of aldo-keto reductase, leptin receptor overlapping transcript, clusterin, and ubiquinone biosynthesis monooxygenase COQ6.

FIG. 12 is screen capture of the genomic location of LPAR-1 from ENSEMBL Chicken Contigview.

FIG. 13 is a table showing identification of a SNP in the Lysophosphatidic acid receptor-1 5′ upstream region.

FIG. 14 is a gel showing confirmation off genotyping by Locked Nucleic Acid Primer PCR.

FIG. 15 depicts screen capture of the results from the transcription factor binding site search using TRANSFAC.

FIG. 16 is another gel showing genotyping of the F₂ generation.

### DETAILED DESCRIPTION AND PREFERRED EMBODIMENTS

In describing a preferred embodiment of the invention specific terminology will be resorted to for the sake of clarity. However, the invention is not intended to be limited to the specific terms so selected, and it is to be understood that each specific term includes all technical equivalents which operate in a similar manner to accomplish a similar purpose.

Fat line and Lean line chickens were produced by inseminating hens with pooled semen from each line (eight pools; seven hens per semen pool). Each hen's eggs were marked and incubated; chicks were sexed, wing-banded, and vaccinated against Marek's disease at hatch. Males (87 Fat, and 102 Lean) were reared together in 4.4×3.9 meter floor pens under a standard heat program. The chickens were fed ad libitum a mashed diet for the first few days, a pelleted starter diet for the first three weeks and then a growing diet up to week 11; water was freely available. Light cycles were 24 hours for the first two days and then 14 hours light/10 hours dark.

Pituitary glands from 8 birds in each line (1 bird per semen pool/different hen) were extracted at weeks 1, 3, 5, and 7, snap-frozen in liquid nitrogen, and stored at −75° C. Birds were weighed and blood was drawn before sacrifice by cervical dislocation. Fat pad was excised and weighed, and other tissues were collected (hypothalamus, breast muscle, liver, and abdominal fat) for use in other studies. The F0 generation used to produce the F2 intercross consisted of thirty animals: four males and thirteen females from the Lean line, and five males and eight females from the Fat line. Fat males were then bred to Lean females, and vice versa, to produce the F1 generation. Five males and fifty females of the F1 generation animals were kept to produce the F2 generation. Three of the F1 males had Fat sires and Lean dams; two had Lean sires and Fat dams. Thirty of the F1 females were from Fat sires and Lean dams; twenty females were from Lean sires and Fat dams. Six hundred thirty-seven F2 animals (332 females and 305 males) were able to provide biometric data. The F0 and the F2 intercross generations were reared under the same conditions and sacrificed at 9 weeks of age. Blood was drawn and body weight and abdominal fat pad weight were measured.

RNA Extraction and Amplification

Total RNA was extracted from individual chicken pituitaries using Qiagen RNeasy mini-prep kits according to the manufacturer's protocols and quantified by absorbance at 260 nm. RNA quality was assessed using the Agilent Bioanalyzer (Agilent Technologies, Palo Alto, Calif.) at the University of Maryland Microarray Core Facility and replacement samples were extracted if the RNA was of low quality. A drawback of using the chicken pituitary as the tissue of interest for microarray analysis is its small size and therefore low content of RNA. To ensure sufficient quantities of RNA for microarray analysis, a variation of the Eberwine procedure was used to generate amplified RNA (aRNA) (Van Gelder, von Zastrow et al. 1990; Luo, Salunga et al. 1999; Porter and Ellestad 2005).

A reverse transcriptase (Superscript II, Invitrogen, Carlsbad, Calif.) was used with a poly-dT primer with a 5′ T7 RNA polymerase promoter sequence (5′-GGCCAGTGAATTGTAATACGACTCACTATAGGGAGGCGGT24-3′ (SEQ ID NO:1); Affymetrix, Santa Clara, Calif.) to transcribe 0.5 ìg of total RNA into first strand cDNA. After RNaseH digestion, DNA polymerase I synthesized the second strand using the digested RNA as primers and the first strand as template; DNA ligase joined the second strand fragments together, and T4 polymerase polished any single stranded overhangs to form blunt-ended double-stranded cDNA. The cDNA was then extracted with phenol-chloroform in a phase-lock centrifuge tube (Eppendorf, Westbury, N.Y.), washed in a Microcon-30 (Millipore, Billerica, Mass.) spin column, and dried down in a vacuum centrifuge.

In vitro transcription of aRNA off of the cDNA was performed using Ambion's (Austin, Tex.) T7 MEGASCRIPT kit as per the manufacturer's directions. Amplified RNA was then phenol-chloroform extracted using a phase-lock centrifuge tube (Eppendorf) and purified by centrifugation through a Spin Column-30 (Sigma, St. Louis, Mo.). The aRNA was then quantified by absorbance at 260 nm and with the RIBOGREEN RNA Quantification kit (Molecular Probes), and visualized by ethidium bromide staining after electrophoresis in an agarose-formaldehyde gel. Note: The RNA amplification procedure has been previously validated in our laboratory. Pooled total RNA and RNA amplified from that pool were hybridized in replicate to 5K Chicken Neuroendocrine System microarrays (GEO accession no. 1744), and the mean log2-transformed raw pixel intensities from each spot were found to be highly correlated (r2=0.96) between the total RNA and the aRNA (Ellestad and Porter 2005).

Labeling and Hybridization of Microarrays

The labeling and hybridization of 1 μg of target aRNA to the Del-Mar 14K Chicken Integrated Systems microarrays (GEO accession no. 1731) was performed by the Microarray Core Facility, Center for Biosystems Research at the University of Maryland, College Park. Target aRNA (1 μg) was reversed transcribed using random primers into cDNA containing a dTTP analog that has a reactive amino allyl group [5-(3-aminoallyl)dUTP] (Ambion). After purification, cDNA was labeled with the ester form of the fluorophores Cy3 and Cy5 (Amersham, Piscataway, N.J.), which link to the amino allyl group.

cDNA from each of the pituitaries was labeled with Cy3, and a pooled reference sample was labeled with Cy5. The pooled reference sample was created from equal amounts of all of the aRNA samples from the experiment. The labeling reactions were purified to remove unincorporated dye, hybridized to the microarrays overnight at 42° C., and then washed with increasingly stringent sodium citrate saline solutions. After washing, slides were scanned by the Facility's 418 confocal laser (Affymetrix) at 550 nm for Cy3 and 650 nm for Cy5. For each slide, a TIFF image file was generated for each fluorophore and saved.

The Institute for Genomic Research (TIGR) makes available a suite of free software for the analysis of microarray data (Saeed, Sharov et al. 2003). The TIFF files were loaded into Spotfinder (version 2.2.4), an image processing program, to visualize the overlaid hybridization scans and to quantify pixel intensities of the spots. The software creates a grid that overlays each spot on the slide within a square cell. The grid for the 14K slides consists of forty-eight 20×20-cell blocks arranged in 4 columns by 12 rows; this grid arrangement matches the printing pattern of the spots. For each hybridization, the Cy3 scan was loaded into channel A and the Cy5 scan into channel B. The slides were analyzed using the Otsu thresholding algorithm option with the spot size parameters set at a minimum of 3 and a maximum of 22.5. The quality control filter was used and the flagged values and raw data were kept. The data generated by Spotfinder analysis were saved as MEV files and exported into Microarray data Analysis System (MIDAS; version 2.18), TIGR's data normalization software, using the data directory mode. This allowed normalization of all the data in a single processing step.

Parameters that were applied were using flags and background checking for both channels; signal-to-noise ratio threshold was set to 3.0. These parameters rejected flagged spots that were saturated, not detected, malformed, or had a background greater than the spot intensity; the background checking parameter keeps only the spots whose intensities are 3 times the background for both channels. Spots that failed to pass these criteria were excluded from downstream analysis. Cy3 spot intensities were normalized by block with the LOWESS algorithm using a smoothing parameter of 0.33, and normalization was followed by standard deviation regularization by block and then by slide using Cy5 (pooled aRNA) as the reference channel. The output was saved as MEV files.

Statistical Analysis

Two-way analysis of variance of the normalized data using Statistical Analysis System software version 8.02 (SAS Institute, Cary, N.C.) was used to identify differentially expressed spots and to trim spots that did not exhibit sufficient changes to be of proximate interest. Only spots with at least 2 replicates for all ages in both lines were examined. The log₂ ratio of normalized Cy3:raw Cy5 was analyzed to detect significant differences (p<0.05) between lines, among ages, and interactions between lines and ages. Spots that did not show any significant differences were excluded from further analysis. The next criterion for further analysis was that the fold-spread of the least squares means of a spot across all ages and lines must be ≧0.68 (log₂ ratio). The final trimming step excluded any spots that did not have intensities greater than the 8 salmon sperm DNA control spots on each array for both Cy3 and Cy5. Three hundred and eighty-six genes were kept for further analysis.

Cluster Analysis

GeneCluster 2 software (http://www.broad.mit.edu/cancer/software/genecluster2/gc2.html) was used to cluster and visualize genes with similar expression patterns by self organizing maps (SOMs) analysis. SOMs use a clustering algorithm that imposes partial structure on a dataset while also reflecting some of the natural structure of the dataset by an iterative classification of data points into nodes, or clusters, which are easy to visually interpret (Tamayo, Slonim et al. 1999). The fat and lean lines were analyzed separately. Genes were classified into 30 clusters with the geometry of the nodes being a 6×5 grid using the default parameters with the exception that the number of iterations was increased to 500,000. To simplify confirmation of gene expression patterns by qRTPCR, 7 expression profiles were defined as follows: 1) gene expression always greater in the Fat line than Lean line, 2) gene expression always greater in the Lean line than Fat line, 3) early age gene expression (weeks 1 and 3) greater in Lean line than Fat line, 4) late age gene expression (weeks 5 and 7) greater in Lean line than Fat line, 5) early age gene expression greater in Fat line than Lean line, 6) late age gene expression greater in Fat line than Lean line, and 7) no difference in gene expression between the 2 lines.

Marker Analysis

GeneCluster 2 software was also used to identify “marker” genes whose up or down regulation is most correlated with the Fat or Lean lines. Twenty-five markers per line were determined at 1-, 3-, and 5-weeks of age using both the signal to noise ratio [(μₐ−μ_{b})/(δₐ+δ_{b})] and the t-test statistic [(μₐ−μ_{b})/√(δₐ²+δ_{b}²)] as the distance metric (μ is the mean per class and δ is the standard deviation per class). Genes with missing replicates were not included in the analysis

Verification of Gene Expression

Expression profiles of microarray gene expression were confirmed by 2-step quantitative reverse transcription PCR (qRT-PCR). Before qRT-PCR, the gel picture of the PCR product that was spotted on the microarray was inspected to ascertain whether PCR amplification of the cDNA library clone produced a clean, single band. Since a DNAse digestion was not performed on the RNA extracted from the pituitaries, genomic contamination in the qRT-PCR reaction was a concern. To design PCR primers, the sequence of the EST clone that was printed on the microarray (http://www.chickest.udel.edu/Cogburn_CAP3_DB) was BLASTed against the chicken genome using ENSEMBL. After confirming that the sequence was within an EST or mRNA, the entire expressed sequence was used to design primer pairs that spanned at least one intron. The lack of a single sharp melting peak of the PCR product indicates probable genomic contamination (although it could be due to alternatively spliced cDNAs). Two genes from each of the 7 expression profiles were verified by qRT-PCR. qRT-PCR for the 14 genes on each of the 32 total RNA samples and a no reverse transcriptase negative control reaction were performed in duplicate. The RNA for the no enzyme control was from the pooled reference total RNA sample. The first step of the qRT-PCR was performed as for the first step of the RNA amplification procedure above except that oligo-dT primer (5′-CGGAATTCTTTTTTTTTTTTTTTTTTTTV-3′) (SEQ ID NO:2), SigmaGenosys, Houston, Tex.) was used. Real time-PCR using Qiagen's Quantitect SYBR Green PCR kits quantified the cDNA from these reactions, along with a water negative control. This required 28 specific primer pairs that were designed using Primer Express software (version 2.0, Applied BioSystems).

The parameters for primer design were a primer length of 18-30 nucleotides spaced 115-130 base pairs apart, a G/C content of 40-60%, and a melting temperature of 58-60° C. The primers were generally targeted to the 3′ end of the gene sequence due to the fact that dT-primed reverse transcription preferentially transcribes mRNA sequences localized in the 3′ end. The real-time 2-step PCR was done in an ICYCLER thermocycler (BioRad). One microliter of cDNA was used as template, and primers were at a final concentration of 300 nM. Thermocycler parameters were an initial enzyme activation incubation for 15 minutes at 95° C., 40 cycles of denaturation at 95° C. for 10 seconds then annealing and extension for 45 seconds at 55° C., and a final denaturation at 95° C. and extension step at 55° C. for one minute each. Primer sequences used are given in Table 1 in the appendix.

Identification of Markers for Genes of Interest

Twelve genes whose expression patterns were confirmed by qRT-PCR were chosen for further analysis. The DNA sequences of the microarray clones for each of the 12 genes were BLASTed against the chicken genome to determine genomic location using ENSEMBL. ENSEMBL GeneSeqView was used to display SNPs in the genomic sequence within 5000 base pairs (bp) upstream of the first exon. Primers were designed that would generate PCR products of about 1000 bp in length containing as many SNPs as possible. Genomic DNA was phenol-chloroform extracted from ˜100 μl of blood taken from F0 animals. Seventeen primer pairs were designed for the 12 genes. The parameters for primer design were a primer length of 18-30 nucleotides spaced 500-1000 base pairs apart, a G/C content of 40-60%, and a melting temperature of 58-60° C. One microliter of genomic DNA (not quantified) was used as starting template, and primers were at a concentration of 200 nM in the reaction. Thermocycler parameters were initial denaturation of 3 minutes at 95° C.; 35 cycles of denaturation at 95° C. for 1 minute, annealing at 55° C. for 1 minute, and then extension for 1 minute at 72° C.; and a final extension step at 72° C. for 7 minutes each. PCR was used to amplify genomic DNA from 22 F0 animals (4 males and 7 females from each line). Primer sequences used are given in Table 2.

Genomic Sequencing

PCR products from the 17 reactions per 22 F0 animals were submitted in 96-well format to the High-Throughput Genomics Unit (HTGU), Department of Genome Sciences, University of Washington for sequencing using the forward primers. The PCR reactions were subjected to clean-up using exonuclease/shrimp alkaline phosphatase by HTGU and sequenced using Applied Biosystems' (Foster City, Calif.) BigDye terminator v3.1 Cycle sequencing kit and a 3730xl DNA analyzer (Applied Biosystems). SNPs were identified by assembling the sequences into contigs using the CONTIGEXPRESS feature in the Vector NTI software package (Invitrogen).

Microarray Analysis

Gene expression profiles in the anterior pituitary were characterized using cDNA microarrays representing greater than 14,000 genes. Pituitaries were extracted from Fat and Lean birds at 1-, 3-, 5-, and 7-weeks of age and total RNA isolated and amplified. Four replicate samples for each strain and age were labeled and hybridized to the Del-Mar 14K Chicken Integrated Systems microarrays (GEO accession no. GPL1731) for a total of 32 samples. The raw data was first subjected to LOWESS normalization using MIDAS and then each slide was subjected to standard deviation regularization by block within slide and the across all slides. Two-way ANOVA (SAS) of log2 ratios was used to detect significant (p<0.05) differences by line, age, and the line-by-age interaction.

The inventors found that there were 1150 significantly different genes between the 2 lines, and 339 of these genes exhibited greater than 0.68-fold differences in their log2 ratios (highest group mean at least 160% of the lowest group mean). One thousand four hundred twenty nine (1429) genes were significantly different with respect to age and of these, 583 exhibited fold changes greater than 0.68 in the log2 ratio. There were 145 genes that significantly differed in their line-by-age interaction, and 62 of these exhibited greater than 0.68-fold differences. It is known that gene expression changes with age. Of the 386 genes with significant differences by line, or for line-by-age interaction, with at least a 0.68-fold change in their log2 ratios and n≧2 for each experimental group were kept for further analysis (Tables 3 and 4).

Cluster Analysis

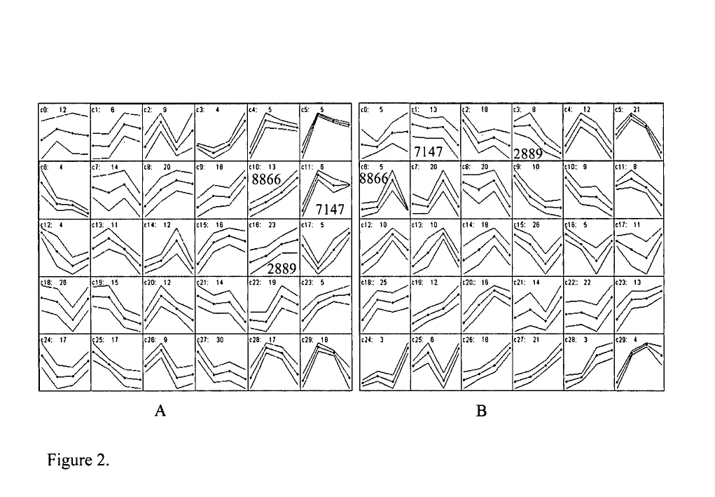

GeneCluster 2 software was used to cluster and visualize genes with similar expression patterns by self-organizing maps (SOMs) analysis. Genes were classified into 30 clusters with the geometry of the nodes being a 6×5 grid. The Lean and Fat lines were analyzed separately. Three genes which represent different expression patterns between the Lean and Fat lines have been identified by microarray spot number (FIG. 2).

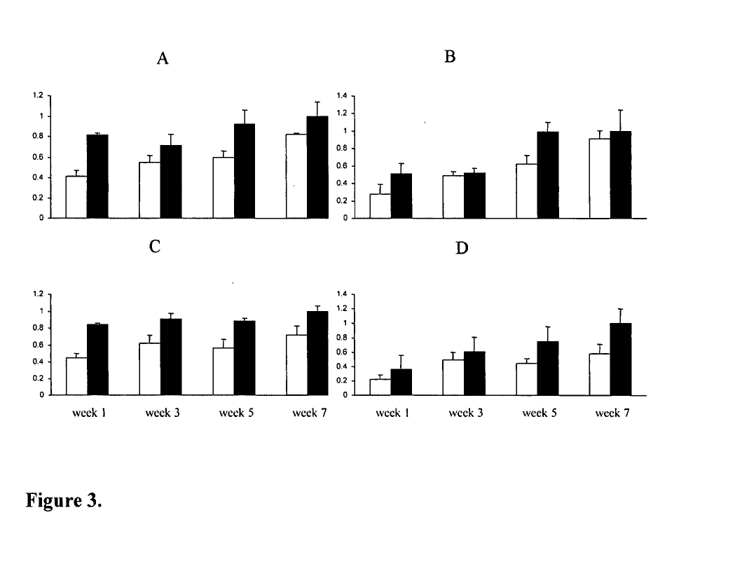

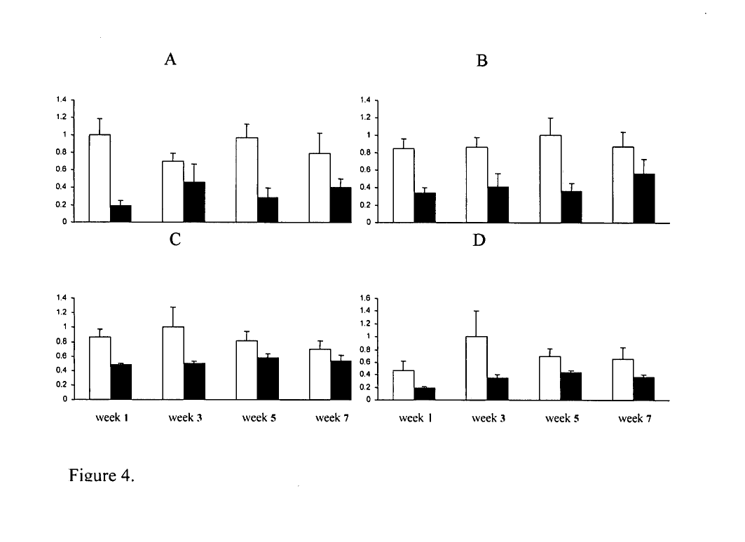

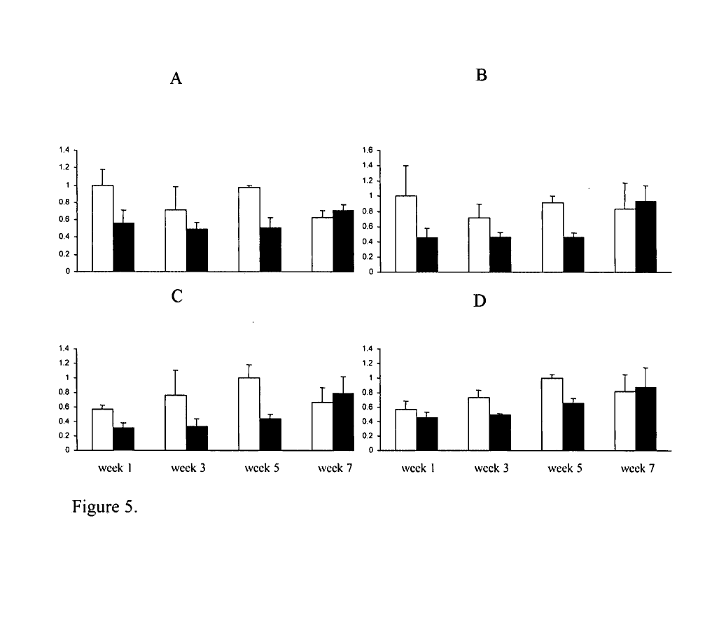

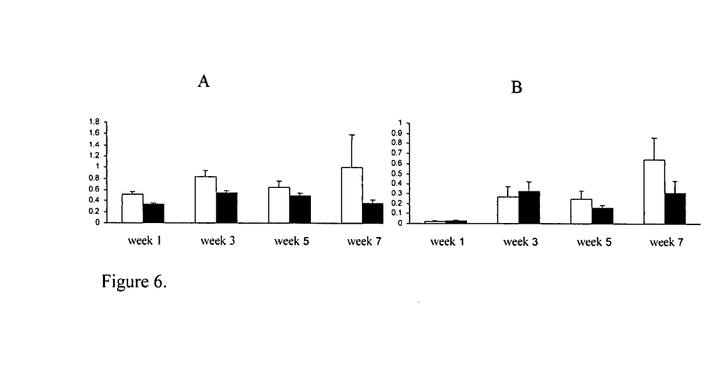

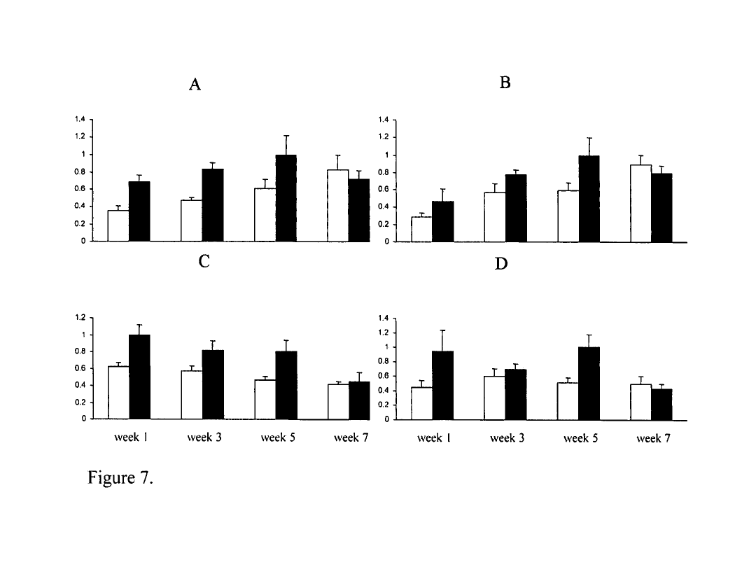

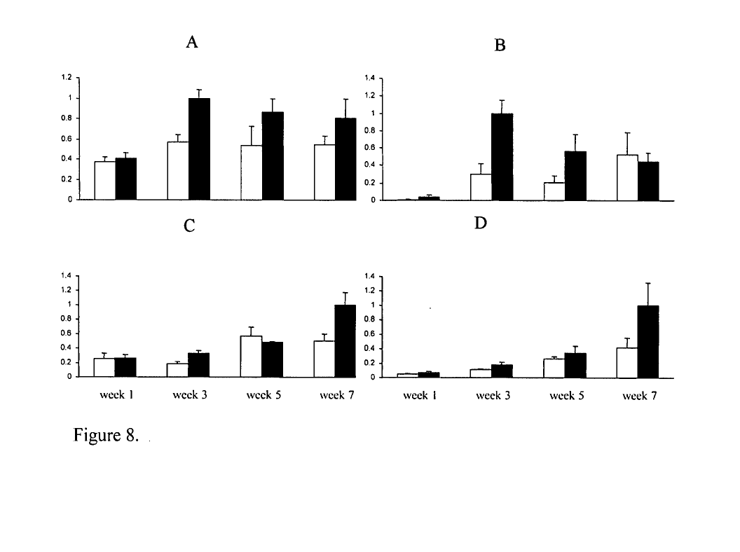

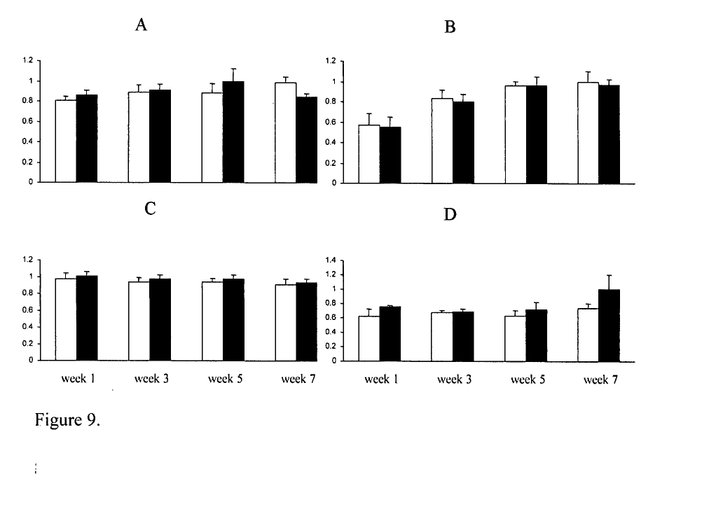

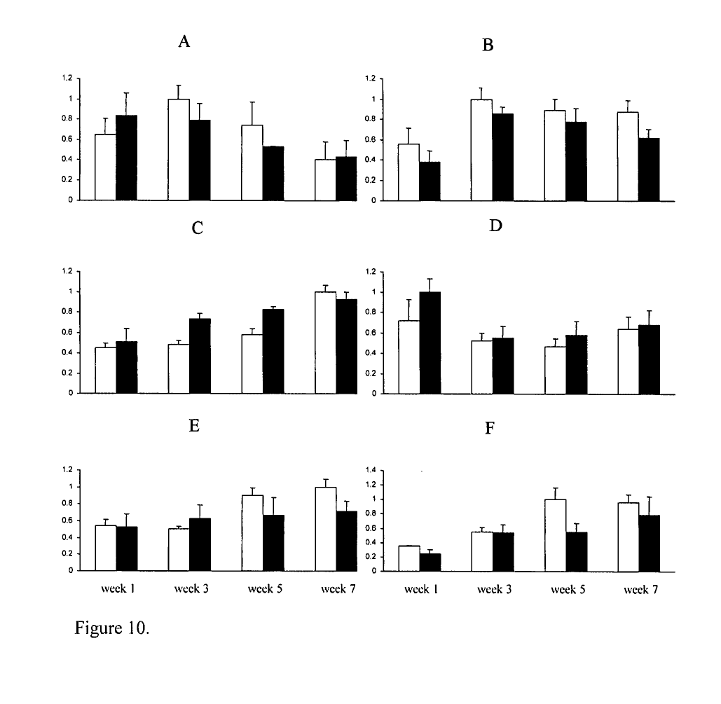

The inventors found that redundancy was apparent in the clusters. To simplify confirmation of gene expression patterns by qRT-PCR, 7 expression profiles were defined using genes that had differences in gene expression between the 2 lines as follows: 1) gene expression always greater in the Fat line than Lean line (FIG. 3); 2) gene expression always greater in the Lean line than Fat line (FIG. 4); 3) early age gene expression (weeks 1 and 3) greater in Lean line than Fat line (FIG. 5); 4) late age gene expression (weeks 5 and 7) greater in Lean line than Fat line (FIG. 6); 5) early age gene expression greater in Fat line than Lean line (FIG. 7); 6) late age gene expression greater in Fat line than Lean line (FIG. 8); and 7) no difference in gene expression between the 2 lines (FIG. 9). Although GH, POMC, and TSH were not significantly different in the microarray analysis, qRT-PCR was performed for those genes (FIG. 10).

Identification of Marker Genes

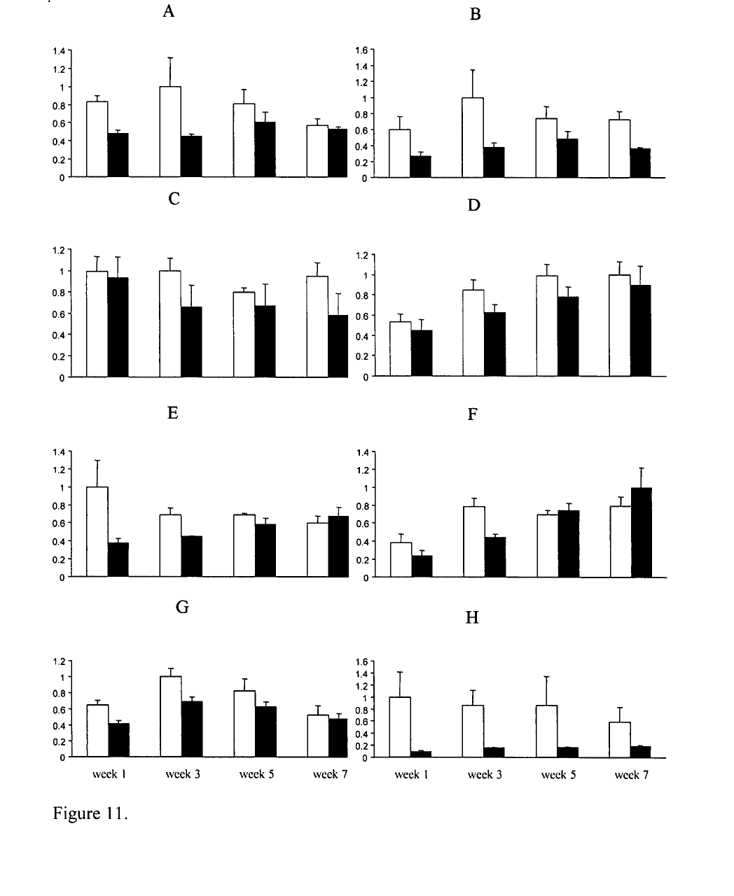

GENECLUSTER 2 software was used to identify the 25 genes most correlated with each strain at 1-, 3-, and 5-weeks of age using both the signal-to-noise ratio and the t-test as the distance metric between the means of the genes in the 2 classes (Lean and Fat). A total of 12 genes were chosen as candidate genes. The 12 genes were either up-regulated (0.68-fold log2 ratios) or were identified in the marker analysis at weeks 1 and 3. The sole exception was Ubiquinone biosynthesis monooxygenase COQ6 (GEO no. 44.3.14), which looked to be highly up-regulated in the Lean line as determined by qRT-PCR. Gene expression levels for eight of the candidate genes were already verified by qRT-PCR (see figures above). In addition to ubiquinone biosynthesis monooxygenase COQ6, Aldo-keto reductase (GEO no. 5.17.9), Leptin receptor overlapping transcript (GEO no. 15.3.6), and Clusterin (GEO no. 20.8.17) were chosen as candidates and expression profiles for these candidates were also verified by qRT-PCR (FIG. 11).

Identification and Genotyping of SNPs

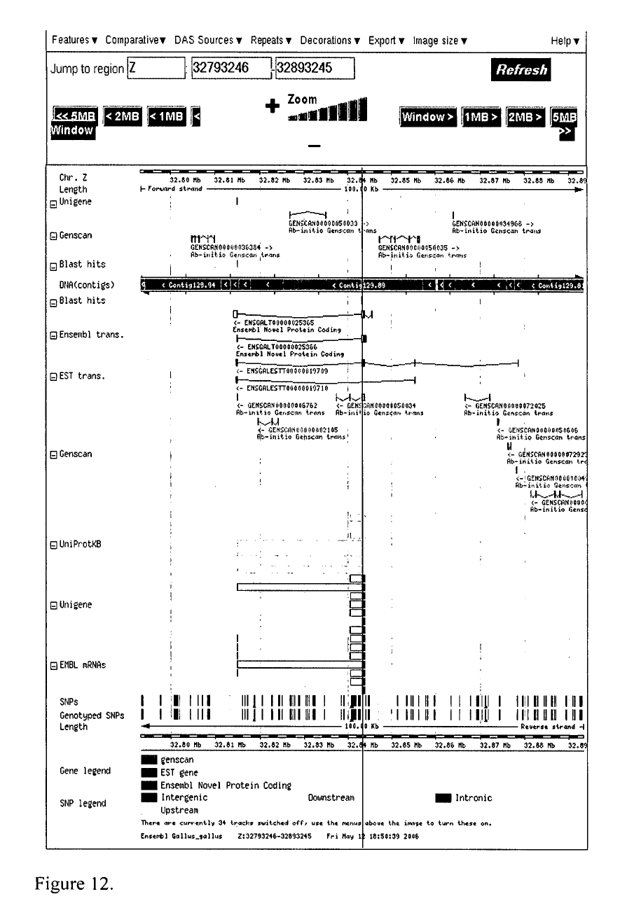

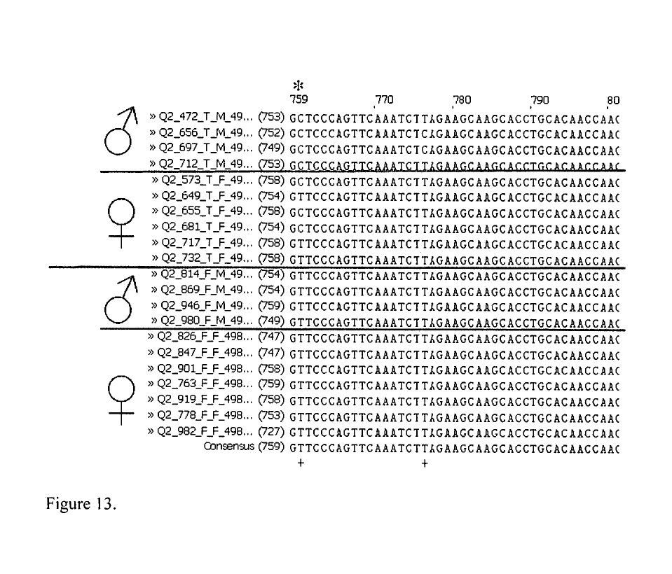

Genomic sequences of the candidate genes were located within the chicken genome using the online database ENSEMBL (http://www.ensembl.org/Gallus_gallus/index.html), which also shows the location of known SNPs (FIG. 12). Regions of up to a 1000 bp containing known SNPs located within 5000 bp upstream of the first exon of 12 genes whose expression patterns were verified by qRTPCR were chosen for sequencing to identify polymorphisms (e.g. SNPs). Although regulatory elements may be located throughout a gene, the upstream region was chosen as a systematic approach to identifying polymorphisms. The sole exception was Leptin receptor overlapping transcript, which was sequenced in the 3′ untranslated region of the mRNA that contained numerous SNPs. Seventeen genomic regions of the 12 candidate genes were amplified by PCR performed on genomic DNA from the F0 generation (4 males and 7 females from each line), and the products were sequenced by the High-Throughput Genomics Unit (HTGU), Department of Genome Sciences, Univ. of Washington. The sequences that were obtained were assembled into contigs using Vector NTI software and examined for SNPs (see FIG. 13 for a representative example). A SNP had to be detected in at least 4 animals within a line to be considered for further analysis. There were 11 SNPs in 5 genes identified that met this criterion.

Confirmation of Sequencing Results

The inventors chose LPAR-1 to be further investigated as a candidate marker for two reasons. First, LPAR-1 exhibited a large difference in SNP frequency within the F0 generation between the two lines. Second, LPAR-1 is known to be involved with cell differentiation, including that of adipocytes (Pages, Girard et al. 2000). However, LPAR-1 was chosen for further investigation due to the fact that it is directly involved in regulating adipocyte differentiation (Pages, Daviaud et al. 2001; Simon, Daviaud et al. 2005).

LPA is a phospholipid found in the serum and is produced by the hydrolysis of cell surface phosphatidic acid by phospholipase A2 or lysophosphatidylcholine by autotaxin/lysophospholipase D (Guo, Kasbohm et al. 2006). Many biological effects are mediated by LPA through multiple G-protein coupled receptor pathways which include stimulation of PLC, inhibition of adenyly cyclase, and Ras-MAPK (Moolenaar, van Meeteren et al. 2004). For example, LPA reduces triglyceride uptake and expression of lipogenesis genes in preadipocytes; removal of LPA from culture medium induces preadipocytes to differentiate. LPAR-1 knockout mice become significantly fatter than wild-type mice but without any difference in food intake-a similar situation to what is seen in the FL and LL. LPAR-1 is close to, but not included within a known QTL for abdominal fat on chromosome Z (flanking markers LEI0111-LEI0075, position 127 cM, genomic location 22950349-31399653) (Ikeobi, Woolliams et al. 2002).

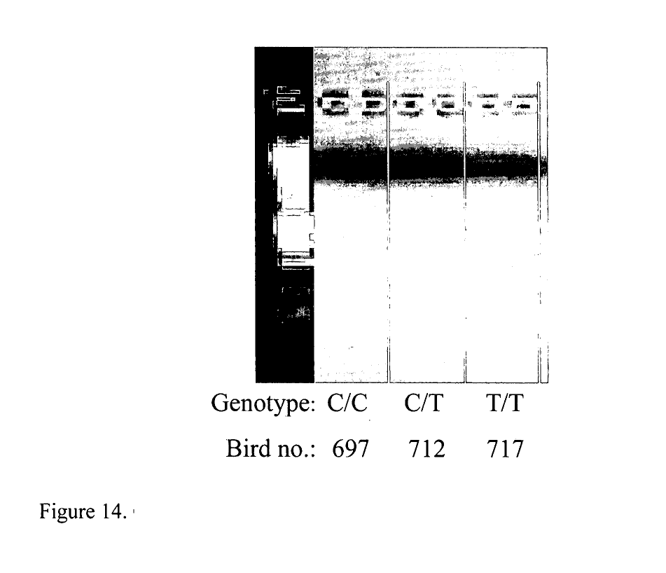

The frequency of the T/C SNP was significantly different between the 2 lines (p≦0.001, Fisher's Exact Test). To confirm that the sequencing results were accurate, the investigators performed PCR using allele-specific locked nucleic acid (LNA) primers. PCR primers with LNA at their 3′-ends are highly specific and can be used to discriminate between two SNPs (Johnson, Haupt et al. 2004). Genotype was determined by the presence or absence of an allele-specific PCR band using the SNP-specific LNA primers (FIG. 14).

The allele-specific PCR confirmed the results of the sequencing performed by HTGU. The Genotypes of the 22 F0 birds are presented in Table 3. The sequence chromatograms indicated that birds 472 and 712 were heterozygous for the T/C SNP, and this was confirmed by PCR.

Identification of Potential Transcription Factor Binding Sites

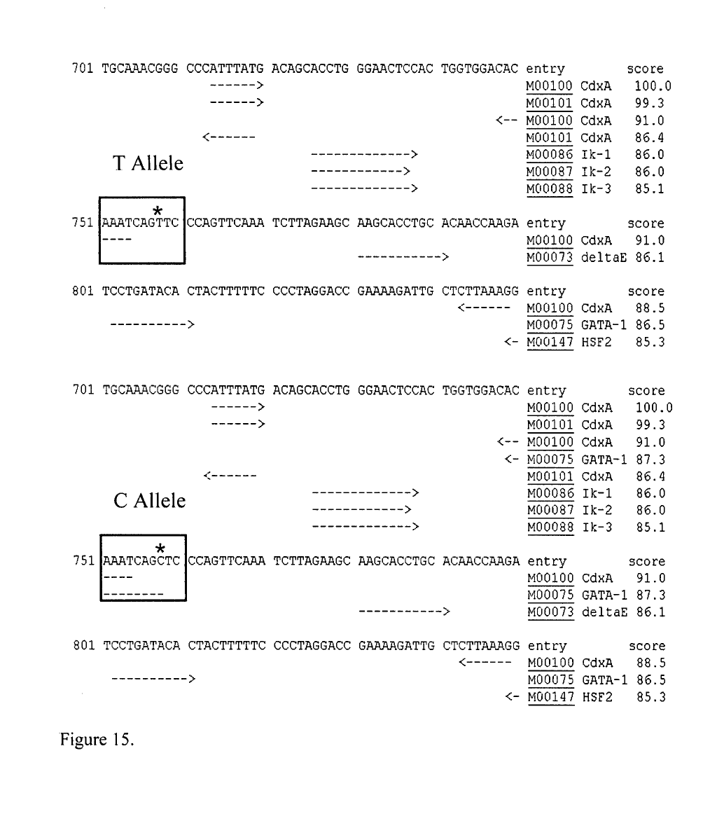

The SNP-specific consensus sequences identified for the LPAR-1 were searched by the inventors for vertebrate transcription factor binding sites using the TRANSFAC (version 1.3) website (http://mbs.cbrc.jp/research/db/TFSEARCH.html; default score threshold=85.0). TRANSFAC is a searchable database that identifies cis-acting elements and their trans-acting factors (Heinemeyer, Wingender et al. 1998). The T-SNP consensus sequence did not show any transcription factor binding sites in the location of the SNP; however the C-SNP introduced a putative GATA-1 binding site (score=87.3) (FIG. 15). When the search score threshold cutoff is lowered to 75.0, an additional putative GATA-2 binding site is introduced in the same sequence (score=79.4) (data not shown).

Genotyping the F2 Generation for the T/C SNP

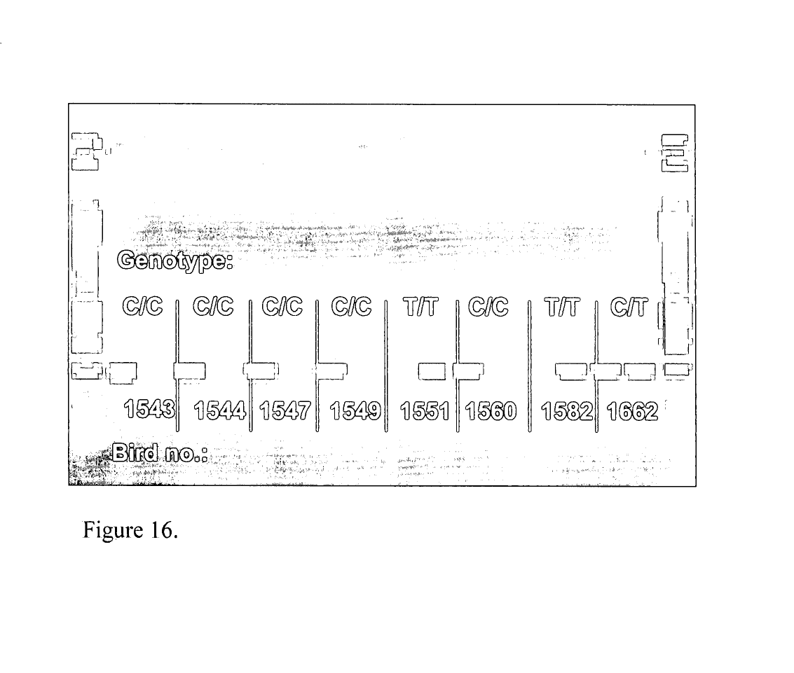

The F0 Lean and Fat lines were intercrossed to produce a F2 generation, and abdominal fat percentage was measured at 9 weeks (n=637). The F2 generation has a normal distribution of abdominal body fat percentage (data not shown). The animals in the tails of the abdominal body percentage distribution (48 leanest males, 48 leanest females, 48 fattest males, and 48 fattest females) were genotyped by LNA PCR (FIG. 16; Table 4). Since the C-SNP introduces a putative GATA transcription factor binding site, animals were categorized into C-SNP positive (C/C and C/T) or negative (TT) genotypes (Table 5). A binomial generalized linear mixed model with a logit link function (PROC GLIMMIX; SAS) was used to test the association of genotypes with the tails of the distributions. PROC GLIMMIX “fits statistical models to data with correlations or nonconstant variability and where the response is not necessarily normally distributed” (SAS Institute 2005). There was a significant association between the C-SNP negative genotype (TT) and the fat tail (p<0.05, n=189).

The inventors completed the genotyping of 399 animals from the F2 population made from the intercross of the Fat and Lean chicken lines. It was found that the percentage of abdominal fat was normally distributed in these animals and ranged from 0.51% to 6.38% of body weight (a range of 5.87%). The mean values for the three genotypes, CC, CT, and TT, were 2.67%, 2.79%, and 3.14%, respectively. These differences were significant at P<0.05. Therefore, the identified SNP in the LPAR1 gene accounts for differences in abdominal fat amounting to 0.47% of body weight. This means that this one SNP statistically accounts for 8% of the total range of values for percent abdominal fat in the population. All of this applies only to males. There was no effect in the females.

The approach taken was successful in identifying a polymorphism upstream of the first exon of the LPAR-1 gene that was significantly associated with adiposity.

TABLE 1
Identification of SNPs in candidate genes. Spot is the microarray spot ID, Gene is the gene name of highest scoring BLASTX hit,
Sequence identifies the SNP, Genome Location is Chromosome: bp to bp, Lean and Fat contain the ratio of animals with each SNP.

| Spot | Gene | Sequence | Gemonic Location | Lean | Fat |
|---|---|---|---|---|---|
| 3341 | Superoxide dismutase | TCCGTGTGCTT(C/T)GGTGGAGGAC | 4: 73905577 to 73905596 | 10C:0T | 3C:7T |
| 3341 | Superoxide dismutase | AATAATAACCT(A/G)AATGATCTAA | 4: 73905410 to 73905431 | 3A:7G | 9G:1A |
| 3341 | Superoxide dismutase | TTTCTACATTA(C/T)GTATTTAGAT | 4: 73905368 to 73905389 | 2C:8T | 9C:1T |
| 4989 | LPA receptor-1 | GGTGGACACAAATCAG(T/C)TCCCAGTTCAAATCTT | Z: 32846253 to 32846285 | 3T:7C | 10T:0C |
| 2889 | Aldo-keto reductase | AAAGAATTCAATCCA(A/T)AATACAGAATTATGG | 1: 59105862 to 59105892 | 1A:10T | 9A:2T |
| 2889 | Aldo-keto reductase | TCAGCACACATA(G/A)CAGCTGTTGAAATG | 1: 59105582 to 59105608 | 6G:5A | 10G:1A |
| 10070 | Glypican | CTGAATGTTCCCCT(A/C)TGGAAATACAGCCC | 4: 3774193 to 3774221 | 6A:5C | 11A:0C |
| 10070 | Glypican | CAGCAAGCAGTCCTG(T/C)TGTACGACTGCATG | 4: 3774280 to 3774309 | 5T:6C | 11T:0C |
| 8866 | Syndecan | TTCTCCTTTAACCAGA(G/C)CAGTTCCCTGATCTG | 3: 99791473 to 99791504 | 5G:6C | 11G:0C |
| 8866 | Syndecan | AATGGCTCCCCAGGG(C/T)GGTGGGCACAGCTCC | 3: 99791109 to 99791139 | 4C:7T | 9C:2T |
| 8866 | Syndecan | CAGCTCCGAGCTGCC(G/A)GAGCTCAAGGAGCA | 3: 99791086 to 99791115 | 5G:6A | 11G:0A |

TABLE 2
Primer sequences for the amplification of F₀ genomic DNA. (GEO platform
GPL1731 no., gene name, forward sequence, reverse sequence).

| GEO no. | Gene name | Forward primer | Reverse primer |
|---|---|---|---|
| 3.1.3 | IgJ | GGCCAGGGATTCCGAAATT | CCTCTGTGATGCTCCTTCACATTA |
| 5.17.9 | Aldo-keto reductase | GAAGTGGTCAGCACACATAGCAG | CCGAGTTTACACGTCCCCTC |
| 5.17.9 | Aldo-keto reductase | TTTGCCCAAATGTAAGGAGAGAGGC | GGTCTTTGCTTTTATGGCTCCAGCT |
| 12.2.1 | Superoxide dismutase | CTTGTGTGCAGGCTTTGGGGAAA | GAAACAAAACTGTGTGCTATGGGGA |
| 12.2.1 | Superoxide dismutase | TAAGGACACACCTTCTTGCTGCTCG | TCTCGGCATAAGAAAAGGGTGAAGA |
| 16.1.20 | Regulator of G-protein | GCTCAGACCGAGAGGCATCT | GCTCCCCTTCCGACAGCTATATAG |
| | signaling 5 | | |
| 14.3.9 | LPA receptor-1 | TGAATAGGTGTCGGCTGTAGAAGCA | TGCTCTGCTGGTGTAAAGGATTCTG |
| 14.3.9 | LPA receptor-1 | TCGTCAGTGCTTGCAGTTCTAAA | TGGAGTAAGGAACCGGACCAA |
| 15.3.6 | Leptin receptor | TGCCTGATCCTGCACGTATC | TCACATCAAGTATTAGTGCACGCA |
| | overlapping transcript | | |
| 20.8.17 | Clusterin | TGGTGGATTCCCATGTATGCTTTC | CAACCTGACCCTGTCCAATGAAGG |
| 18.10.6 | Early activation | GCTCAGTTCAGCGAGGCTCAT | CCCGGCATTACCTCACTGAGA |
| | antigen CD69 | | |
| 24.11.6 | Syndecan-1 precursor | ACAGTCCCTCATCAGTTATGTAGGC | GGATCCCGTTAGCTACTGTAGGTGT |
| 24.11.6 | Syndecan-1 precursor | GTGTCAGCATCCCAGGAACC | AGGACAAGCAGTAGCGCTGC |
| 28.6.10 | Glypican-3 precursor | GGAGAAGGGAGAAGCTCTTTGCAAT | AAGAAAAAAGCATTCCTGGAAAGGC |
| 41.3.17 | Plasma glutamate | CCGTTTGTTCTACAGGTTCAACCA | CAGCTATCTCATTTCTGATGCCTTC |
| | carboxypeptidase | | |
| 44.3.14 | Ubiquinone biosynthesis | CCAAACACCAGAGCTCCTAAGAC | TGCCAATTGAAACTTGCTAGCA |
| | monooxgenase COQ6 | | |
| 44.3.14 | Ubiquinone biosynthesis | CCGTTTGTTCTACAGGTTCAACCA | CAGCTATCTCATTTCTGATGCCTTC |
| | monooxgenase COQ6 | | |

TABLE 3
Candidate genes chosen for sequencing.

| | GEO | |
|---|---|---|
| Spot | no. | BLAST X |
| 43 | 3.1.3 | Immunoglobulin J polypeptide, linker protein for |
| | | immunoglobulin alpha and mu polypeptides |
| 2889 | 5.17.9 | aldo-keto reductase |
| 3341 | 12.2.1 | Extracellular superoxide dismutase [Cu—Zn] |
| | | precursor (EC 1.15.1.1) (EC-SOD) |
| 4880 | 16.1.20 | Regulator of G-protein signaling 5 |
| 4989 | 14.3.9 | Lysophosphatidic acid receptor Edg-2 (LPA |
| | | receptor 1) (LPA-1) |
| 5006 | 15.3.6 | leptin receptor overlapping transcript |
| 7037 | 20.8.17 | clusterin (complement lysis inhibitor, SP-40,40, |
| | | sulfated glycoprotein 2, testosterone-repressed |
| | | prostate message 2, apolipoprotein J) |
| 7147 | 18.10.6 | Early activation antigen CD69 (Early T-cell |
| | | activation antigen p60) (GP32/28) (Leu-23) |
| | | (MLR-3) (EA1) (BL-AC/P26) (Activation inducer |
| | | molecule) (AIM) |
| 8866 | 24.11.6 | Syndecan-1 precursor (SYND1) (CD138 antigen) |
| 10070 | 28.6.10 | Glypican-3 precursor (Intestinal protein OCI-5) |
| | | (GTR2-2) (MXR7) |
| 16177 | 41.3.17 | Plasma glutamate carboxypeptidase |
| 16234 | 44.3.14 | Ubiquinone biosynthesis monooxgenase COQ6 |

TABLE 4
Identification of SNPs in candidate genes. Spot is the microarray spot ID, Gene is the gene name of highest scoring BLASTX hit.
Sequence identifies the SNP, Genome Location is Chromosome: bp to bp. Lean and Fat contain the ratio of animals with each SNP.

| Spot | Gene | Sequence | Genomic Location | Lean | Fat |
|---|---|---|---|---|---|
| 3341 | Superoxide dismutase | TCCGTGTGCTT(C/T)GGTGGAGGAC | 4: 73905577 to 73905596 | 10C:0T | 3C:7T |
| 3341 | Superoxide dismutase | AATAATAACCT(A/G)AATGATCTAA | 4: 73905410 to 73905431 | 3A:7G | 9G:1A |
| 3341 | Superoxide dismutase | TTTCTACATTA(C/T)GTATTTAGAT | 4: 73905368 to 73905389 | 2C:8T | 9C:1T |
| 4989 | LPA receptor-1 | GGTGGACACAAATCAG(T/C)TCCCAGTTCAAATCTT | Z: 32846253 to 32846285 | 3T:7C | 10T:0C |
| 2889 | Aldo-keto reductase | AAAGAATTCAATCCA(A/T)AATACAGAATTATGG | 1: 59105862 to 59105892 | 1A:10T | 9A:2T |
| 2889 | Aldo-keto reductase | TCAGCACACATA(G/A)CAGCTGTTGAAATG | 1: 59105582 to 59105608 | 6G:5A | 10G:1A |
| 10070 | Glypican | CTGAATGTTCCCCT(A/C)TGGAAATACAGCCC | 4: 3774193 to 3774221 | 6A:5C | 11A:0C |
| 10070 | Glypican | CAGCAAGCAGTCCTG(T/C)TGTACGACTGCATG | 4: 3774280 to 3774309 | 5T:6C | 11T:0C |
| 8866 | Syndecan | TTCTCCTTTAACCAGA(G/C)CAGTTCCCTGATCTG | 3: 99791473 to 99791504 | 5G:6C | 11G:0C |
| 8866 | Syndecan | AATGGCTCCCCAGGG(C/T)GGTGGGCACAGCTCC | 3: 99791109 to 99791139 | 4C:7T | 9C:2T |
| 8866 | Syndecan | CAGCTCCGAGCTGCC(G/A)GAGCTCAAGGAGCA | 3: 99791086 to 99791115 | 5G:6A | 11G:0A |

TABLE 5
SNP-specific genotypes of the F₀ birds. Bird
ID no., Line, Sex, and Genotype are given.

| Bird no. | Line | Sex | Genotype |
|---|---|---|---|
| 472 | Lean | Male | C/T |
| 573 | Lean | Female | C/C |
| 649 | Lean | Female | T/T |
| 655 | Lean | Female | C/C |
| 656 | Lean | Male | C/C |
| 681 | Lean | Female | C/C |
| 695 | Lean | Female | C/C |
| 697 | Lean | Male | C/C |
| 712 | Lean | Male | C/T |
| 717 | Lean | Female | T/T |
| 732 | Lean | Female | T/T |
| 763 | Fat | Female | T/T |
| 778 | Fat | Female | T/T |
| 814 | Fat | Male | T/T |
| 826 | Fat | Female | T/T |
| 847 | Fat | Female | T/T |
| 869 | Fat | Male | T/T |
| 901 | Fat | Female | T/T |
| 919 | Fat | Female | T/T |
| 946 | Fat | Male | T/T |
| 980 | Fat | Male | T/T |
| 982 | Fat | Female | T/T |

TABLE 6
Genotypes of the animals in the tails of the body fat
percentage distribution. Bird ID, genotype as determine
by LNA PCR, sex, tail, body weight at sacrifice, weight
of abdominal fat pad, body fat percentage.

| | | | | Body weight | Abdominal | Fat |
|---|---|---|---|---|---|---|
| Bird | | | | at 9 weeks | fat pad | yield |
| no. | Genotype | Sex | Tail | (g) | weight (g) | (%) |
| 1292 | CT | Male | Lean | 2388 | 12.16 | 0.509 |
| 1916 | TT | Male | Lean | 2191 | 15.73 | 0.718 |
| 1764 | TT | Male | Lean | 1855 | 17.25 | 0.930 |
| 1190 | CT | Male | Lean | 2237 | 22.90 | 1.024 |
| 1302 | CC | Male | Lean | 2135 | 27.19 | 1.274 |
| 1535 | CC | Female | Lean | 1912 | 26.43 | 1.382 |
| 1202 | TT | Male | Lean | 2323 | 32.49 | 1.399 |
| 1864 | CC | Male | Lean | 2427 | 35.32 | 1.455 |
| 1531 | TT | Male | Lean | 2314 | 34.12 | 1.475 |
| 1193 | TT | Female | Lean | 1726 | 25.57 | 1.481 |
| 1547 | CC | Male | Lean | 2307 | 35.38 | 1.534 |
| 51986 | CT | Male | Lean | 1958 | 30.57 | 1.561 |
| 51896 | TT | Male | Lean | 3089 | 48.39 | 1.567 |
| 51970 | CT | Male | Lean | 2133 | 33.60 | 1.575 |
| 1663 | CT | Male | Lean | 1996 | 31.52 | 1.579 |
| 1739 | TT | Female | Lean | 1559 | 24.73 | 1.586 |
| 1743 | CT | Male | Lean | 2396 | 38.33 | 1.600 |
| 1430 | TT | Male | Lean | 2263 | 36.52 | 1.614 |
| 1815 | TT | Female | Lean | 1943 | 32.40 | 1.668 |
| 1359 | TT | Male | Lean | 2143 | 35.80 | 1.671 |
| 51981 | CT | Male | Lean | 2755 | 46.39 | 1.684 |
| 1465 | TT | Male | Lean | 2420 | 41.16 | 1.701 |
| 1963 | CC | Male | Lean | 2531 | 43.24 | 1.708 |
| 1194 | TT | Female | Lean | 1696 | 29.03 | 1.712 |
| 1549 | CC | Male | Lean | 2563 | 44.34 | 1.730 |
| 51997 | CT | Male | Lean | 2358 | 41.62 | 1.765 |
| 1662 | CT | Male | Lean | 2851 | 50.96 | 1.787 |
| 1551 | TT | Female | Lean | 1948 | 35.61 | 1.828 |
| 1962 | CT | Male | Lean | 2546 | 46.80 | 1.838 |
| 1423 | CT | Male | Lean | 2346 | 43.28 | 1.845 |
| 1650 | CT | Male | Lean | 2420 | 44.82 | 1.852 |
| 1504 | TT | Male | Lean | 2580 | 48.25 | 1.870 |
| 1530 | CT | Male | Lean | 2344 | 44.22 | 1.887 |
| 1568 | CC | Male | Lean | 2314 | 43.66 | 1.887 |
| 51977 | CC | Female | Lean | 1874 | 35.37 | 1.887 |
| 1753 | CT | Male | Lean | 2685 | 50.74 | 1.890 |
| 1539 | TT | Female | Lean | 2018 | 38.17 | 1.891 |
| 1639 | TT | Male | Lean | 2704 | 51.29 | 1.897 |
| 1495 | TT | Male | Lean | 2189 | 41.78 | 1.909 |
| 1528 | CC | Female | Lean | 1309 | 25.03 | 1.912 |
| 1543 | CC | Female | Lean | 2033 | 38.91 | 1.914 |
| 1942 | TT | Male | Lean | 2374 | 45.55 | 1.919 |
| 1494 | TT | Female | Lean | 1651 | 31.68 | 1.919 |
| 1757 | CT | Male | Lean | 2604 | 50.50 | 1.939 |
| 1678 | CC | Male | Lean | 2180 | 42.67 | 1.957 |
| 1164 | TT | Male | Lean | 2448 | 48.24 | 1.971 |
| 1500 | TT | Male | Lean | 2329 | 46.08 | 1.979 |
| 1859 | CC | Male | Lean | 2452 | 48.52 | 1.979 |
| 1399 | TT | Male | Lean | 1938 | 38.44 | 1.983 |
| 1401 | TT | Male | Lean | 1892 | 37.63 | 1.989 |
| 1571 | CC | Male | Lean | 2569 | 51.39 | 2.000 |
| 1973 | CC | Male | Lean | 1928 | 38.57 | 2.001 |
| 1965 | TT | Female | Lean | 1857 | 37.39 | 2.013 |
| 1805 | TT | Male | Lean | 2450 | 49.39 | 2.016 |
| 1200 | TT | Male | Lean | 2276 | 46.55 | 2.045 |
| 1924 | TT | Male | Lean | 2430 | 49.92 | 2.054 |
| 51987 | CC | Female | Lean | 2085 | 42.92 | 2.059 |
| 1715 | CT | Male | Lean | 2426 | 50.10 | 2.065 |
| 1589 | CC | Male | Lean | 2550 | 52.95 | 2.076 |
| 1869 | CC | Male | Lean | 2234 | 46.42 | 2.078 |
| 1703 | TT | Female | Lean | 1525 | 31.80 | 2.085 |
| 1282 | CC | Male | Lean | 2596 | 54.17 | 2.087 |
| 1582 | TT | Female | Lean | 1817 | 39.22 | 2.159 |
| 1544 | CC | Female | Lean | 2084 | 44.99 | 2.159 |
| 1668 | CC | Female | Lean | 1863 | 40.74 | 2.187 |
| 1676 | CC | Female | Lean | 1872 | 41.22 | 2.202 |
| 1802 | TT | Female | Lean | 1322 | 29.84 | 2.257 |
| 1675 | CC | Female | Lean | 1477 | 33.52 | 2.269 |
| 1971 | TT | Female | Lean | 1748 | 39.73 | 2.273 |
| 51933 | TT | Female | Lean | 2289 | 52.96 | 2.314 |
| 1348 | TT | Female | Lean | 1683 | 39.14 | 2.326 |
| 51916 | TT | Female | Lean | 1846 | 43.17 | 2.339 |
| 1346 | TT | Female | Lean | 1718 | 40.58 | 2.362 |
| 1384 | TT | Female | Lean | 1679 | 39.66 | 2.362 |
| 1115 | CT | Female | Lean | 2349 | 55.59 | 2.367 |
| 1221 | TT | Female | Lean | 1953 | 46.40 | 2.376 |
| 1163 | TT | Female | Lean | 1639 | 39.10 | 2.386 |
| 1677 | TT | Female | Lean | 1921 | 45.99 | 2.394 |
| 1457 | TT | Female | Lean | 2139 | 53.38 | 2.496 |
| 1950 | TT | Female | Lean | 1833 | 46.13 | 2.517 |
| 1533 | TT | Female | Lean | 1792 | 45.42 | 2.535 |
| 1964 | TT | Female | Lean | 2166 | 54.96 | 2.537 |
| 1553 | TT | Female | Lean | 1893 | 48.24 | 2.548 |
| 1161 | TT | Female | Lean | 1790 | 46.08 | 2.574 |
| 1588 | . | Female | Lean | 1790 | 46.39 | 2.592 |
| 1470 | TT | Female | Lean | 1398 | 36.33 | 2.599 |
| 1310 | CC | Female | Lean | 1409 | 36.68 | 2.603 |
| 1959 | TT | Female | Lean | 1699 | 44.29 | 2.607 |
| 1987 | TT | Female | Lean | 1715 | 45.05 | 2.627 |
| 1651 | . | Female | Lean | 1754 | 46.25 | 2.637 |
| 1199 | CC | Female | Lean | 2083 | 55.17 | 2.649 |
| 1156 | TT | Female | Lean | 1724 | 45.77 | 2.655 |
| 1824 | CC | Female | Lean | 2007 | 53.49 | 2.665 |
| 51950 | TT | Female | Lean | 1943 | 52.13 | 2.683 |
| 1649 | TT | Female | Lean | 1925 | 51.73 | 2.687 |
| 1212 | CC | Female | Lean | 1774 | 47.81 | 2.695 |
| 51886 | TT | Male | Fat | 2719 | 95.43 | 3.510 |
| 1503 | TT | Male | Fat | 2092 | 73.64 | 3.520 |
| 1667 | CT | Male | Fat | 2290 | 80.85 | 3.531 |
| 1814 | TT | Male | Fat | 2659 | 94.49 | 3.554 |
| 1511 | TT | Male | Fat | 2498 | 89.66 | 3.589 |
| 1852 | CC | Male | Fat | 2796 | 100.65 | 3.600 |
| 1480 | TT | Male | Fat | 2541 | 91.71 | 3.609 |
| 1449 | CT | Male | Fat | 2508 | 90.83 | 3.622 |
| 1940 | TT | Male | Fat | 2070 | 75.35 | 3.640 |
| 1762 | CC | Male | Fat | 2967 | 108.10 | 3.643 |
| 1467 | CT | Male | Fat | 2540 | 92.76 | 3.652 |
| 1437 | TT | Male | Fat | 2475 | 90.46 | 3.655 |
| 51893 | TT | Male | Fat | 2779 | 101.65 | 3.658 |
| 51888 | TT | Male | Fat | 2832 | 104.20 | 3.679 |
| 1801 | TT | Male | Fat | 2332 | 86.59 | 3.713 |
| 1182 | CC | Male | Fat | 2222 | 83.10 | 3.740 |
| 1779 | TT | Male | Fat | 2790 | 104.95 | 3.762 |
| 1055 | TT | Male | Fat | 2549 | 95.89 | 3.762 |
| 1958 | TT | Male | Fat | 1801 | 68.65 | 3.812 |
| 51877 | CT | Male | Fat | 2876 | 110.23 | 3.833 |
| 1821 | TT | Male | Fat | 2602 | 101.08 | 3.885 |
| 1478 | TT | Male | Fat | 2573 | 100.28 | 3.897 |
| 1429 | CC | Male | Fat | 2478 | 97.05 | 3.916 |
| 1914 | TT | Male | Fat | 2539 | 99.47 | 3.918 |
| 1045 | TT | Male | Fat | 2606 | 103.48 | 3.971 |
| 1358 | TT | Male | Fat | 2380 | 95.65 | 4.019 |
| 51900 | TT | Male | Fat | 2840 | 114.47 | 4.031 |
| 1464 | TT | Male | Fat | 2549 | 103.49 | 4.060 |
| 1825 | CC | Male | Fat | 2466 | 100.87 | 4.090 |
| 1696 | TT | Male | Fat | 2604 | 106.59 | 4.093 |
| 51879 | CT | Male | Fat | 2956 | 121.96 | 4.126 |
| 1717 | CT | Male | Fat | 2546 | 105.24 | 4.134 |
| 51942 | CT | Male | Fat | 3022 | 125.34 | 4.148 |
| 1823 | CC | Male | Fat | 2099 | 87.80 | 4.183 |
| 51982 | CC | Male | Fat | 2815 | 118.13 | 4.196 |
| 1169 | TT | Male | Fat | 1957 | 82.24 | 4.202 |
| 51946 | TT | Male | Fat | 3021 | 127.47 | 4.219 |
| 1096 | TT | Male | Fat | 2301 | 98.37 | 4.275 |
| 1217 | CC | Male | Fat | 2046 | 87.83 | 4.293 |
| 1862 | CC | Male | Fat | 2424 | 105.21 | 4.340 |
| 1440 | TT | Male | Fat | 2101 | 91.99 | 4.378 |
| 1923 | TT | Male | Fat | 1990 | 88.15 | 4.430 |
| 1442 | TT | Male | Fat | 2543 | 117.87 | 4.635 |
| 1466 | CT | Male | Fat | 2668 | 125.05 | 4.687 |
| 1172 | TT | Female | Fat | 1926 | 90.44 | 4.696 |
| 1585 | TT | Female | Fat | 2024 | 95.55 | 4.721 |
| 1101 | TT | Female | Fat | 1764 | 83.35 | 4.725 |
| 1778 | TT | Female | Fat | 1987 | 93.95 | 4.728 |
| 1718 | TT | Female | Fat | 1922 | 91.08 | 4.739 |
| 1364 | . | Female | Fat | 2176 | 103.12 | 4.739 |
| 51940 | CC | Female | Fat | 2299 | 109.03 | 4.742 |
| 51954 | CC | Female | Fat | 2233 | 106.10 | 4.751 |
| 1139 | TT | Female | Fat | 1933 | 92.69 | 4.795 |
| 1436 | CT | Male | Fat | 2894 | 139.46 | 4.819 |
| 1927 | TT | Female | Fat | 1929 | 93.02 | 4.822 |
| 1137 | TT | Female | Fat | 2066 | 99.87 | 4.834 |
| 1949 | TT | Female | Fat | 1762 | 85.50 | 4.852 |
| 1912 | CC | Female | Fat | 1947 | 94.55 | 4.856 |
| 1438 | CT | Male | Fat | 2361 | 114.78 | 4.861 |
| 1796 | TT | Female | Fat | 2211 | 107.70 | 4.871 |
| 1443 | TT | Female | Fat | 2125 | 103.57 | 4.874 |
| 51889 | TT | Female | Fat | 2208 | 107.76 | 4.880 |
| 1167 | TT | Female | Fat | 2149 | 105.14 | 4.893 |
| 51880 | CC | Female | Fat | 2230 | 109.37 | 4.904 |
| 51911 | TT | Male | Fat | 2970 | 146.64 | 4.937 |
| 51910 | TT | Female | Fat | 2213 | 109.61 | 4.953 |
| 1697 | TT | Female | Fat | 2045 | 101.39 | 4.958 |
| 1756 | CC | Female | Fat | 2321 | 115.22 | 4.964 |
| 1656 | TT | Female | Fat | 2219 | 110.38 | 4.974 |
| 1352 | TT | Female | Fat | 1903 | 94.92 | 4.988 |
| 1141 | TT | Female | Fat | 2069 | 103.40 | 4.998 |
| 51901 | TT | Female | Fat | 1841 | 92.47 | 5.023 |
| 51985 | TT | Female | Fat | 2265 | 114.28 | 5.045 |
| 51904 | TT | Female | Fat | 2062 | 104.79 | 5.082 |
| 1413 | TT | Female | Fat | 2262 | 115.45 | 5.104 |
| 1724 | TT | Female | Fat | 1990 | 101.71 | 5.111 |
| 1768 | TT | Female | Fat | 2192 | 112.12 | 5.115 |
| 51913 | TT | Female | Fat | 2183 | 111.93 | 5.127 |
| 51891 | TT | Female | Fat | 2227 | 114.32 | 5.133 |
| 1979 | TT | Female | Fat | 1790 | 92.49 | 5.167 |
| 1693 | TT | Female | Fat | 1928 | 101.90 | 5.285 |
| 1694 | TT | Female | Fat | 2316 | 122.64 | 5.295 |
| 1166 | TT | Female | Fat | 2095 | 111.14 | 5.305 |
| 1340 | TT | Female | Fat | 1955 | 103.77 | 5.308 |
| 1357 | TT | Male | Fat | 2713 | 148.87 | 5.487 |
| 1560 | CC | Female | Fat | 2121 | 117.22 | 5.527 |
| 1103 | CC | Female | Fat | 2044 | 113.01 | 5.529 |
| 51960 | CC | Female | Fat | 2278 | 127.43 | 5.594 |
| 1863 | TT | Female | Fat | 1634 | 91.69 | 5.611 |
| 1933 | TT | Female | Fat | 2072 | 116.39 | 5.617 |
| 51927 | TT | Female | Fat | 2283 | 128.62 | 5.634 |
| 1713 | CC | Female | Fat | 2086 | 117.72 | 5.643 |
| 51871 | CC | Female | Fat | 2122 | 120.38 | 5.673 |
| 1812 | TT | Female | Fat | 2154 | 123.81 | 5.748 |
| 51968 | TT | Female | Fat | 2138 | 136.25 | 6.373 |
| 1695 | TT | Female | Fat | 2278 | 145.27 | 6.377 |

TABLE 7
Number of animals from the tails of the abdominal
body fat percentage. The CC/CT genotypes contain
the GATA transcription factor binding site.

| Genotype | CC/CT | TT |
|---|---|---|
| Lean tail animals | 43 | 51 |
| Fat tail animals | 29 | 66 |

### BIBLIOGRAPHY

Ahmed, S. and S. Harvey (2002). “Ghrelin: a hypothalamic GH-releasing factor in domestic fowl (Gallus domesticus).” J Endocrinol 172(1): 117-25.
Altan, O., A. Pabuccuoglu, et al. (2003). “Effect of heat stress on oxidative stress, lipid peroxidation and some stress parameters in broilers.” Br Poult Sci 44(4): 545-50.
Angioni, A. R., A. Lania, et al. (2005). “Effects of chronic retinoid administration on pituitary function.” J Endocrinol Invest 28(11): 961-4.
Bailleul, B., I. Akerblom, et al. (1997). “The leptin receptor promoter controls expression of a second distinct protein.” Nucleic Acids Res 25(14): 2752-8.
Bartov, I., L. S. Jensen, et al. (1980). “Effect of corticosterone and prolactin on fattening in broiler chicks.” Poult Sci 59(6): 1328-34.
Beccavin, C., B. Chevalier, et al. (2001). “Insulin-like growth factors and body growth in chickens divergently selected for high or low growth rate.” J Endocrinol 168(2): 297-306.
Bernot, A., R. Zoorob, et al. (1994). “Linkage of a new member of the lectin supergene family to chicken Mhc genes.” Immunogenetics 39(4): 221-9.
Buonomo, F. C., T. J. Lauterio, et al. (1987). “Effects of insulin-like growth factor I (IGF-I) on growth hormone-releasing factor (GRF) and thyrotropin-releasing hormone (TRH) stimulation of growth hormone (GH) secretion in the domestic fowl (Gallus domesticus).” Gen Comp Endocrinol 66(2): 274-9.
Buyse, J., E. Decuypere, et al. (1987). “Effect of corticosterone on circulating concentrations of corticosterone, prolactin, thyroid hormones and somatomedin C and on fattening in broilers selected for high or low fat content.” J Endocrinol 112(2): 229-37.
Buyse, J., A. Vanderpooten, et al. (1994). “Pulsatility of plasma growth hormone and hepatic growth hormone receptor characteristics of broiler chickens divergently selected for abdominal fat content.” Br Poult Sci 35(1): 145-52.
Cahaner, A., Z. Nitsan, et al. (1986). “Weight and fat content of adipose and nonadipose tissues in broilers selected for or against abdominal adipose tissue.” poultry Science 65: 215-222.
Carlborg, O., L. Jacobsson, et al. (2006). “Epistasis and the release of genetic variation during long-term selection.” Nat Genet 38(4): 418-20.
Carre, W., X. Wang, et al. (2006). “Chicken Genomics Resource Sequencing and Annotation of 35,407 ESTs from Single and Multiple Tissue cDNA Libraries and CAP3 Assembly of a Chicken Gene Index.” Physiol Genomics.
Carsia, R. V., H. Weber, et al. (1988). “Protein malnutrition in the domestic fowl induces alterations in adrenocortical function.” 122: 673-680.
Carsia, R. V., H. Weber, et al. (1986). “Corticotropin-releasing factor stimulates the release of adrenocorticotropin from domestic fowl pituitary cells.” Endocrinology 118(1): 143-8.
Charles, M. A., T. L. Saunders, et al. (2006). “Pituitary-specific gata2 knockout: effects on gonadotrope and thyrotrope function.” Mol Endocrinol 20(6): 1366-77.
Cogburn, L. A., X. Wang, et al. (2004). “Functional genomics in chickens: development of integrated-systems microarrays for transcriptional profiling and discovery of regulatory pathways.” Comp Funct Genom 5: 253-261.
Cogburn, L. A., X. Wang, et al. (2003). “Systems-wide chicken DNA microarrays, gene expression profiling, and discovery of functional genes.” Poult Sci 82(6): 939-51.
Crosas, B., E. Cederlund, et al. (2001). “A vertebrate aldo-keto reductase active with retinoids and ethanol.” J Biol Chem 276(22): 19132-40.
Daval, S., S. Lagarrigue, et al. (2000). “Messenger RNA levels and transcription rates of hepatic lipogenesis genes in genetically lean and fat chickens.” Genet Sel Evol 32(5): 521-31.
De Groef, B., N. Goris, et al. (2003). “Corticotropin-releasing hormone (CRH)-induced thyrotropin release is directly mediated through CRH receptor type 2 on thyrotropes.” Endocrinology 144(12): 5537-44.
de Silva, H. V., W. D. Stuart, et al. (1990). “A 70-kDa apolipoprotein designated ApoJ is a marker for subclasses of human plasma high density lipoproteins.” J Biol Chem 265(22): 13240-7.
Decuypere, E., J. Buyse, et al. (1987). “Effects of hyper- or hypothyroid status on growth, adiposity and levels of growth hormone, somatomedin C and thyroid metabolism in broiler chickens.” Reprod Nutr Dev 27(2B): 555-65.
Decuypere, E., P. Van As, et al. (2005). “Thyroid hormone availability and activity in avian species: a review.” Domest Anim Endocrinol 29(1): 63-77.
Deeb, N. and S. J. Lamont (2002). “Genetic architecture of growth and body composition in unique chicken populations.” J Hered 93(2): 107-18.
DeGroef, B., K. L. Geris, et al. (2003). “Involvement of thyrotropin-releasing hormone receptor, somatostatin receptor subtype 2 and corticotropin-releasing hormone receptor type 1 in the control of chicken thyrotropin secretion.” Molecular and Cellular Endocrinology 203: 33-39.
DeGroef, B., S. V. H. Grommen, et al. (2004). “Cloning and tissue distribution of the chicken type 2 corticotropin-releasing hormone receptor.” General and Comparative Endocrinology 138: 89-95.
Denver, R. J. and S. Harvey (1991). “Thyroidal inhibition of chicken pituitary growth hormone: alterations in secretion and accumulation of newly synthesized hormone.” J Endocrinol 131(1): 39-48.
Douaire, M., N. L. Fur, et al. (1992). “Identifying genes involved in the variability of genetic fatness in the growing chicken.” Poultry Science 71: 1911-1920.
Duggan, D. J., M. Bittner, et al. (1999). “Expression profiling using cDNA microarrays.” Nature Genetics 21: 33-37.
Dupont, J., J. Chen, et al. (1999). “Metabolic differences between genetically lean and fat chickens are partly attributed to the alteration of insulin signaling in liver.” J Nutr 129(11): 1937-44.
Ellestad, L. and T. Porter (2005). Gene expression profiling in the developing neuroendocrine system of the chick. Avian Endocrinology. A. Dawson and P. Sharp. New Dehli, Narosa Publishing House.
Ellestad, L. E., W. Carre, et al. (2006). “Gene expression profiling during cellular differentiation in the embryonic pituitary gland using cDNA microarrays.” Physiol Genomics 25(3): 414-25.
Emara, M. G. and H. Kim (2003). “Genetic markers and their application in poultry breeding.” Poult Sci 82(6): 952-7.
Fotouhi, N., C. N. Karatzas, et al. (1993). “Identification of growth hormone DNA polymorphisms which respond to divergent selection for abdominal fat content in chickens.” Theor. Appl. Genet. 85: 931-936.
Geris, K. L., E. D'Hondt, et al. (1999). “Thyrotropin-releasing hormone concentrations in different regions of the chicken brain and pituitary: an ontogenetic study.” Brain Res 818(2): 260-6.
Geris, K. L., G. Meeussen, et al. (2000). “Distribution of somatostatin in the brain and of somatostatin and thyrotropin-releasing hormone in peripheral tissues of the chicken.” Brain Res 873(2): 306-9.
Gin, P., A. Y. Hsu, et al. (2003). “The Saccharomyces cerevisiae COQ6 gene encodes a mitochondrial flavin-dependent monooxygenase required for coenzyme Q biosynthesis.” J Biol Chem 278(28): 25308-16.
Gingras, R., C. Richard, et al. (1999). “Purification, cDNA cloning, and expression of a new human blood plasma glutamate carboxypeptidase homologous to N-acetyl-aspartyl-alpha-glutamate carboxypeptidase/prostate-specific membrane antigen.” J Biol Chem 274(17): 11742-50.
Golub, T. R., D. K. Slonim, et al. (1999). “Molecular classification of cancer: class discovery and class prediction by gene expression monitoring.” Science 286(5439): 531-7.
Gregory, C. C. and T. E. Porter (1997). “Cloning and sequence analysis of a cDNA for the beta subunit of chicken thyroid-stimulating hormone.” Gen Comp Endocrinol 107(2): 182-90.
Griffin, H. D. (1993). Metabolic and endocrine control of genetic variation in fat deposition in growing chickens. Avian endocrinology. P. J. Sharp. Bristol, Journal of Endocrinology Ltd.: 285-296.
Griffin, H. D. and C. Goddard (1994). “Rapidly growing broiler (meat-type) chickens: their origin and use for comparative studies of the regulation of growth.” Int J Biochem 26(1): 19-28.
Griffin, H. D., K. Guo, et al. (1992). “Adipose tissue lipogenesis and fat deposition in leaner broiler chickens.” J Nutr 122(2): 363-8.
Grommen, S. V., S. Taniuchi, et al. (2006). “Molecular cloning, tissue distribution and ontogenic thyroidal expression of the chicken thyrotropin receptor.” Endocrinology.
Gududuru, V., K. Zeng, et al. (2006). “Identification of Darmstoff analogs as selective agonists and antagonists of lysophosphatidic acid receptors.” Bioorg Med Chem Lett 16(2): 451-6.
Guo, R., E. A. Kasbohm, et al. (2006). “Expression and Function of Lysophosphatidic Acid LPA1 Receptor in Prostate Cancer Cells.” Endocrinology In press.
Harvey, S. (1990). “Tri-iodothyronine inhibition of thyrotrophin-releasing hormone-induced growth hormone release from the chicken adenohypophysis in vitro.” J Endocrinol 126(1): 75-81.
Harvey, S., D. Attardo, et al. (1990). “Somatostatin binding to chicken adenohypophysial membranes.” J Mol Endocrinol 4(3): 213-21.
Harvey, S. and J. S. Baidwan (1990). “Thyroidal inhibition of growth hormone secretion in fowl: tri-iodothyronine-induced down-regulation of thyrotrophin-releasing hormone-binding sites on pituitary membranes.” J Mol Endocrinol 4(2): 127-34.
Harvey, S., R. A. Fraser, et al. (1991). “Growth hormone secretion in poultry.” Critical Reviews in Poultry Biology 3: 239-282.
Havenstein, G. B., P. R. Ferket, et al. (2003). “Carcass composition and yield of 1957 versus 2001 broilers when fed representative 1957 and 2001 broiler diets.” Poult Sci 82(10): 1509-18.
Hayashi, H., K. Imai, et al. (1991). “Characterization of chicken ACTH and alpha-MSH: the primary sequence of chicken ACTH is more similar to Xenopus ACTH than to other avian ACTH.” Gen Comp Endocrinol 82(3): 434-43.
Hedley, A. A., C. L. Ogden, et al. (2004). “Prevalence of overweight and obesity among US children, adolescents, and adults, 1999-2002.” Jama 291(23): 2847-50.
Hermier, D. (1997). “Lipoprotein metabolism and fattening in poultry.” J Nutr 127(5 Suppl): 805S-808S.
Hermier, D., A. Quignard-Boulange, et al. (1989). “Evidence of enhanced storage capacity in adipose tissue of genetically fat chickens.” J Nutr 119(10): 1369-75.
Herold, M., H. P. Brezinschek, et al. (1992). “Investigation of ACTH responses of chickens with autoimmune disease.” Gen Comp Endocrinol 88(2): 188-98.
Hillier, L. W., W. Miller, et al. (2004). “Sequence and comparative analysis of the chicken genome provide unique perspectives on vertebrate evolution.” Nature 432(7018): 695-716.
Hood, R. L. (1982). “The cellular basis for growth of the abdominal fat pad in broiler-type chickens.” Poult Sci 61(1): 117-21.
Huang, X. and A. Madan (1999). “CAP3: A DNA sequence assembly program.” Genome Res 9(9): 868-77.
Ikeobi, C. O., J. A. Woolliams, et al. (2002). “Quantitative trait loci affecting fatness in the chicken.” Anim Genet 33(6): 428-35.
Jazet, I. M., H. Pijl, et al. (2003). “Adipose tissue as an endocrine organ: impact on insulin resistance.” Neth J Med 61(6): 194-212.
Jozsa, R., S. Vigh, et al. (1984). “Localization of corticotropin-releasing factor-containing neurons in the brain of the domestic fowl. An immunohistochemical study.” Cell Tissue Res 236(1): 245-8.
Julian, R. J. (2005). “Production and growth related disorders and other metabolic diseases of poultry—a review.” Vet J 169(3): 350-69.
Kacsoh, B. (2000). The adipose organ: appetite, leptin, and obesity. Endocrine Physiology. New York, McGraw-Hill: 657-681.
Kersten, S. (2002). Peroxisome proliferator activated receptors and obesity. European Journal of Pharmacology 440: 223-234.
Kolset, S. O. and M. Salmivirta (1999). “Cell surface heparan sulfate proteoglycans and lipoprotein metabolism.” Cell Mol Life Sci 56(9-10): 857-70.
Lagarrigue, S., F. Pitel, et al. (2006). “Mapping quantitative trait loci affecting fatness and breast muscle weight in meat-type chicken lines divergently selected on abdominal fatness.” Genet Sel Evol 38(1): 85-97.
Lamb, I. C., D. M. Galehouse, et al. (1988). “Chicken growth hormone cDNA sequence.” Nucleic Acids Research 16: 9339.
Leclercq, B. (1983). “The influence of dietary protein content on the performance of genetically lean or fat growing chickens.” Br Poult Sci 24(4): 581-7.
Leclercq, B. (1988). Genetic selection of meat-type chickens for high or low abdominal fat. Leaness in Domestic Birds. B. Lerclercq and C. Whitehead. London, Leaness in Domestic Birds: 25-40.
Leclercq, B., J. Blum, et al. (1980). “Selecting broilers for low or high abdominal fat: Initial observations.” British Poultry Science 21: 107-113.
Leclercq, B. and G. Guy (1991). “Further investigations on protein requirement of genetically lean and fat chickens.” Br Poult Sci 32(4): 789-98.
Leclercq, B., G. Guy, et al. (1988). “Thyroid hormones in genetically lean or fat chickens: effects of age and triiodothyronine supplementation.” Reprod Nutr Dev 28(4A): 931-7.
Leclercq, B., D. Hermier, et al. (1984). “Effects of age and diet on plasma lipid and glucose concentrations in genetically lean or fat chickens.” Reprod Nutr Dev 24(1): 53-61.
Leclercq, B., J. Kouassi-Kouakou, et al. (1985). “Laying performances, egg composition, and glucose tolerance of genetically lean or fat meat-type breeders.” Poult Sci 64(9): 1609-16.
Leclercq, B. and A. Saadoun (1982). “Selecting broilers for low or high abdominal fat: Comparison of energy metabolism of the lean and fat lines.” Poultry Science 61: 1799-1803.
Legrand, P., J. Mallard, et al. (1987). “Hepatic lipogenesis in genetically lean and fat chickens. In vitro studies.” Comp Biochem Physiol B 87(4): 789-92.
Lemarchal, P., P. Beaunez, et al. (1988). “In vivo turnover of triglyceride fatty acid in the adipose tissue of chickens (Gallus domesticus) selected for low or high adiposity.” Comparative biochemistry and physiology. B, Comparative biochemistry 89(2): 227-231.
Leonard, M. W., K. C. Lim, et al. (1993). “Expression of the chicken GATA factor family during early erythroid development and differentiation.” Development 119(2): 519-31.
Luo, L., R. C. Salunga, et al. (1999). “Gene expression profiles of laser-captured adjacent neuronal subtypes.” Nat Med 5(1): 117-22.
Ma, X. M., C. Camacho, et al. (2001). “Regulation of corticotropin-releasing hormone (CRH) transcription and CRH mRNA stability by glucocorticoids.” Cell Mol Neurobiol 21(5): 465-75.
Ma, Y.-Y., X.-F. Qi, et al. (2005). “cDNA microarray reveals signaling pathways involved in hormones expression of human pituitary.” General and Comparative Endocrinology 143: 184-192.
McNabb, A. F. M. (2000). Thyroids. Sturkie's Avian Physiology. G. C. Whittow. San Diego, Academic press: 461-471.
McRory, J. E., R. L. Parker, et al. (1997). “Expression and alternative processing of a chicken gene encoding both growth hormone-releasing hormone and pituitary adenylate cyclase-activating polypeptide.” DNA Cell Biol 16(1): 95-102.
Mendelson, C. R. (2004). Mechanisms of hormone action. Textbook of Endocrine Physiology. J. E. Griffin and S. R. Ojeda. New York, Oxford University Press, Inc.: 120-146.
Moolenaar, W. H., L. A. van Meeteren, et al. (2004). “The ins and outs of lysophosphatidic acid signaling.” Bioessays 26(8): 870-81.
Muchow, M., I. Bossis, et al. (2005). “Ontogeny of pituitary thyrotrophs and regulation by endogenous thyroid hormone feedback in the chick embryo.” J Endocrinol 184(2): 407-16.
Muoio, D. M. and C. B. Newgard (2006). “Obesity-related derangements in metabolic regulation.” Annu Rev Biochem 75: 367-401.
Nelson, D. L. and M. M. Cox (2000). Lehninger Principles of Biochemistry. New York, Worthpublishers.
Nie, L., G. Wu, et al. (2006). “Correlation between mRNA and protein abundance in Desulfovibrio vulgaris: a multiple regression to identify sources of variations.” Biochem Biophys Res Commun 339(2): 603-10.
Nir, I., Z. Nitzan, et al. (1988). Fat deposition in birds. Leaness in Domestic Birds. B. Lerclercq and C. Whitehead. London, Butterworths: 141-174.
Oken, E. and M. W. Gillman (2003). “Fetal origins of obesity.” Obes Res 11(4): 496-506.
Pages, C., D. Daviaud, et al. (2001). “Endothelial differentiation gene-2 receptor is involved in lysophosphatidic acid-dependent control of 3T3F442A preadipocyte proliferation and spreading.” J Biol Chem 276(15): 11599-605.
Pages, G., A. Girard, et al. (2000). “LPA as a paracrine mediator of adipocyte growth and function.” Ann N Y Acad Sci 905: 159-64.
Porter, T. and L. Ellestad (2005). Gene expression profiling in the developing neuroendocrine system of the chicken. Functional Avian Endocrinology. A. Dawson and P. sharp. New Delhi, Narosa Publishing House.
Porter, T. E., L. E. Ellestad, et al. (2006). “Identification of the chicken growth hormone-releasing hormone receptor (GHRH-R) mRNA and gene: regulation of anterior pituitary GHRH-R mRNA levels by homologous and heterologous hormones.” Endocrinology 147(5): 2535-43.
Richards, M. P., S. M. Poch, et al. (2006). “Characterization of turkey and chicken ghrelin genes, and regulation of ghrelin and ghrelin receptor mRNA levels in broiler chickens.” Gen Comp Endocrinol 145(3): 298-310.
Saadoun, A. and B. Leclercq (1983). “Comparison of in vivo fatty acid synthesis of the genetically lean and fat chickens.” Comp Biochem Physiol B 75(4): 641-4.
Saadoun, A. and B. Leclercq (1987). “In vivo lipogenesis of genetically lean and fat chickens: effects of nutritional state and dietary fat.” J Nutr 117(3): 428-35.
Saadoun, A., J. Simon, et al. (1987). “Effect of exogenous corticosterone in genetically fat and lean chickens.” Br Poult Sci 28(3): 519-28.
Saadoun, A., J. Simon, et al. (1988). “Levels of insulin, corticosterone, T3, T4 and insulin sensitivity in fat and lean chickens.” Diabete Metab 14(2): 97-103.
Saeed, A., V. Sharov, et al. (2003). “TM4: a free, open-source system for microarray data management and analysis.” Biotechniques 34(2): 374-8.
SAS Institute (2005). “The GLIMMIX Procedure, November 2005.”
Scanes, C. G., J. A. Proudman, et al. (1999). “Influence of continuous growth hormone or insulin-like growth factor I administration in adult female chickens.” Gen Comp Endocrinol 114(3): 315-23.
Sharp, P. J., R. B. Chiasson, et al. (1979). “Localization of cells producing thyroid stimulating hormone in the pituitary gland of the domestic drake.” Cell and Tissue Research 198: 53-63.
Simon, J., B. Chevalier, et al. (1991). “Normal number and kinase activity of insulin receptors in liver of genetically fat chickens.” J Nutr 121(3): 379-85.
Simon, M. F., D. Daviaud, et al. (2005). “Lysophosphatidic acid inhibits adipocyte differentiation via lysophosphatidic acid 1 receptor-dependent down-regulation of peroxisome proliferator-activated receptor gamma2.” J Biol Chem 280(15): 14656-62.
Spinedi, E., M. Giacomini, et al. (1991). “Changes in the hypothalamo-corticotrope axis after bilateral adrenalectomy: evidence for a median eminence site of glucocorticoid action.” Neuroendocrinology 53 (2): 160-70.
Takahashi, T., T. Iwase, et al. (2000). “Cloning and expression of the chicken immunoglobulin joining (J)-chain cDNA.” Immunogenetics 51(2): 85-91.
Takeuchi, S., K. Teshigawara, et al. (1999). “Molecular cloning and characterization of the chicken pro-opiomelanocortin (POMC) gene.” Biochim Biophys Acta 1450(3): 452-9.
Tamayo, P., D. Slonim, et al. (1999). “Interpreting patterns of gene expression with self-organizing maps: methods and application to hematopoietic differentiation.” Proc Natl Acad Sci USA 96(6): 2907-12.
Tanaka, M., T. Miyazaki, et al. (2003). “Molecular characterization of chicken growth hormone secretagogue receptor gene.” Gen Comp Endocrinol 134(2): 198-202.
Thommes, R. C., J. B. Martens, et al. (1983). “Hypothalamo-adenohypophyseal-thyroid interrelationships in the chick embryo. IV. Immunocytochemical demonstration of TSH in the hypophyseal pars distalis.” Gen Comp Endocrinol 51(3): 434-43.
Tong, Q., G. Dalgin, et al. (2000). “Function of GATA transcription factors in preadipocyte-adipocyte transition.” Science 290(5489): 134-8.
Toogood, A. A., S. Harvey, et al. (2006). “Cloning of the chicken pituitary receptor for growth hormone-releasing hormone.” Endocrinology 147(4): 1838-46.
Van Gelder, R. N., M. E. von Zastrow, et al. (1990). “Amplified RNA synthesized from limited quantities of heterogeneous cDNA.” Proc Natl Acad Sci USA 87(5): 1663-7.
Vandenborne, K., B. De Groef, et al. (2005). “Molecular cloning and developmental expression of corticotropin-releasing factor in the chicken.” Endocrinology 146(1): 301-8.
Vandenborne, K., B. De Groef, et al. (2005). “Corticosterone-induced negative feedback mechanisms within the hypothalamo-pituitary-adrenal axis of the chicken.” J Endocrinol 185(3): 383-91.
Vandenborne, K., S. A. Roelens, et al. (2005). “Cloning and hypothalamic distribution of the chicken thyrotropin-releasing hormone precursor cDNA.” J Endocrinol 186(2): 387-96.
Wang, C. Y., Y. Wang, et al. (2006). “Expression profiles of growth hormone-releasing hormone and growth hormone-releasing hormone receptor during chicken embryonic pituitary development.” Poult Sci 85(3): 569-76.
Wang, D. G., J. B. Fan, et al. (1998). “Large-scale identification, mapping, and genotyping of single-nucleotide polymorphisms in the human genome.” Science 280(5366): 1077-82.
Whitehead, C. C. and H. D. Griffin (1984). “Development of divergent lines of lean and fat broilers using plasma very low density lipoprotein concentration as selection criterion: the first three generations.” Br Poult Sci 25(4): 573-82.
Wong, G. K., B. Liu, et al. (2004). “A genetic variation map for chicken with 2.8 million single-nucleotide polymorphisms.” Nature 432(7018): 717-22.
Yu, J., L. Y. Xie, et al. (1996). “Molecular cloning of a type A chicken corticotropin-releasing factor receptor with high affinity for urotensin I.” Endocrinology 137(1): 192-7.
Zhong, H. and R. R. Neubig (2001). “Regulator of G protein signaling proteins: novel multifunctional drug targets.” J Pharmacol Exp Ther 297(3): 837-45.

The patents, patent applications and references cited herein are all hereby incorporated by reference in their entireties, as if set forth in the instant specification.

Having described the invention, many modifications thereto will become apparent to those skilled in the art to which it pertains without deviation from the spirit of the invention as defined by the scope of the appended claims.

We claim:

1. A genetic marker for determining the adiposity of an animal, wherein an thymidine in position 17 upstream of the first exon of the LPAR-1 gene is characteristic of a lean animal and a cytidine in position of 17 the LPAR-1 gene is characteristic of a obese animal.

2. The genetic marker of claim 1, wherein the animal is a mammal.

3. The genetic marker of claim 1, wherein the animal is a human.

4. A genetic marker for determining the adiposity of poultry, wherein an thymidine in position 17 upstream of the first exon of the LPAR-1 gene is characteristic of lean poultry and a cytidine in position 17 the LPAR-1 gene is characteristic of fat poultry.

5. The genetic marker of claim 4, wherein the type of poultry is chicken.

6. An isolated polynucleotide having the following sequences GGTGGACACAAATCAGTTCCCAGTTCAAATCTT (SEQ ID NO: 56) and GGTGGACACAAATCAGCTCCCAGTTCAAATCTT (SEQ ID NO: 57) associated with polymorphism in the first exon of the LPAR-1 gene.

7.-9. (canceled)

10. A method for identifying a animal having a genotype that is indicative of advantageous meat production traits, comprising the following steps: a) obtaining a biological sample comprising the DNA of said animal; b) analyzing the polymorphism of the first exon of the LPAR-1 gene of said animal, in which the presence of allele T or C of the gene is indicative of high potential for lean or fatty meat production in said animal.

11. The identification method according to claim 10, in which the animal is a member of the poultry family.

12. The identification method of claim 11, in which the animal is a chicken.

13. The identification method of claim 10, wherein step a) comprises isolating the DNA from animal blood cells.

14. The identification method of claim 10, wherein step b) comprises allele-specific amplification and/or detection.

15.-24. (canceled)


SEQUENCE LISTING (57 sequences)

SEQ ID NO: 1 - DNA, 63 bp, Artificial Sequence
  Description of Artificial Sequence Synthetic primer
ggccagtgaattgtaatacgactcactatagggaggcggtttttttttttttttttttttttt

SEQ ID NO: 2 - DNA, 29 bp, Artificial Sequence
  Description of Artificial Sequence Synthetic primer
cggaattcttttttttttttttttttttv

SEQ ID NO: 3 - DNA, 22 bp, Artificial Sequence
  Description of Artificial Sequence Synthetic oligonucleotide
tccgtgtgcttyggtggaggac

SEQ ID NO: 4 - DNA, 22 bp, Artificial Sequence
  Description of Artificial Sequence Synthetic oligonucleotide
aataataacctraatgatctaa

SEQ ID NO: 5 - DNA, 22 bp, Artificial Sequence
  Description of Artificial Sequence Synthetic oligonucleotide
tttctacattaygtatttagat

SEQ ID NO: 6 - DNA, 33 bp, Artificial Sequence
  Description of Artificial Sequence Synthetic oligonucleotide
ggtggacacaaatcagytcccagttcaaatctt

SEQ ID NO: 7 - DNA, 31 bp, Artificial Sequence
  Description of Artificial Sequence Synthetic oligonucleotide
aaagaattcaatccawaatacagaattatgg

SEQ ID NO: 8 - DNA, 27 bp, Artificial Sequence
  Description of Artificial Sequence Synthetic oligonucleotide
tcagcacacatarcagctgttgaaatg

SEQ ID NO: 9 - DNA, 29 bp, Artificial Sequence
  Description of Artificial Sequence Synthetic oligonucleotide
ctgaatgttcccctmtggaaatacagccc

SEQ ID NO: 10 - DNA, 30 bp, Artificial Sequence
  Description of Artificial Sequence Synthetic oligonucleotide
cagcaagcagtcctgytgtacgactgcatg

SEQ ID NO: 11 - DNA, 32 bp, Artificial Sequence
  Description of Artificial Sequence Synthetic oligonucleotide
ttctcctttaaccagascagttccctgatctg

SEQ ID NO: 12 - DNA, 31 bp, Artificial Sequence
  Description of Artificial Sequence Synthetic oligonucleotide
aatggctccccagggyggtgggcacagctcc

SEQ ID NO: 13 - DNA, 30 bp, Artificial Sequence
  Description of Artificial Sequence Synthetic oligonucleotide
cagctccgagctgccrgagctcaaggagca

SEQ ID NO: 14 - DNA, 19 bp, Artificial Sequence
  Description of Artificial Sequence Synthetic primer
ggccagggattccgaaatt

SEQ ID NO: 15 - DNA, 23 bp, Artificial Sequence
  Description of Artificial Sequence Synthetic primer
gaagtggtcagcacacatagcag

SEQ ID NO: 16 - DNA, 25 bp, Artificial Sequence
  Description of Artificial Sequence Synthetic primer
tttgcccaaatgtaaggagagaggc

SEQ ID NO: 17 - DNA, 23 bp, Artificial Sequence
  Description of Artificial Sequence Synthetic primer
cttgtgtgcaggctttggggaaa

SEQ ID NO: 18 - DNA, 25 bp, Artificial Sequence
  Description of Artificial Sequence Synthetic primer
taaggacacaccttcttgctgctcg

SEQ ID NO: 19 - DNA, 20 bp, Artificial Sequence
  Description of Artificial Sequence Synthetic primer
gctcagaccgagaggcatct

SEQ ID NO: 20 - DNA, 25 bp, Artificial Sequence
  Description of Artificial Sequence Synthetic primer
tgaataggtgtcggctgtagaagca

SEQ ID NO: 21 - DNA, 23 bp, Artificial Sequence
  Description of Artificial Sequence Synthetic primer
tcgtcagtgcttgcagttctaaa

SEQ ID NO: 22 - DNA, 20 bp, Artificial Sequence
  Description of Artificial Sequence Synthetic primer
tgcctgatcctgcacgtatc

SEQ ID NO: 23 - DNA, 24 bp, Artificial Sequence
  Description of Artificial Sequence Synthetic primer
tggtggattcccatgtatgctttc

SEQ ID NO: 24 - DNA, 21 bp, Artificial Sequence
  Description of Artificial Sequence Synthetic primer
gctcagttcagcgaggctcat

SEQ ID NO: 25 - DNA, 25 bp, Artificial Sequence
  Description of Artificial Sequence Synthetic primer
acagtccctcatcagttatgtaggc

SEQ ID NO: 26 - DNA, 20 bp, Artificial Sequence
  Description of Artificial Sequence Synthetic primer
gtgtcagcatcccaggaacc

SEQ ID NO: 27 - DNA, 25 bp, Artificial Sequence
  Description of Artificial Sequence Synthetic primer
ggagaagggagaagctctttgcaat

SEQ ID NO: 28 - DNA, 24 bp, Artificial Sequence
  Description of Artificial Sequence Synthetic primer
ccgtttgttctacaggttcaacca

SEQ ID NO: 29 - DNA, 23 bp, Artificial Sequence
  Description of Artificial Sequence Synthetic primer
ccaaacaccagagctcctaagac

SEQ ID NO: 30 - DNA, 24 bp, Artificial Sequence
  Description of Artificial Sequence Synthetic primer
ccgtttgttctacaggttcaacca

SEQ ID NO: 31 - DNA, 24 bp, Artificial Sequence
  Description of Artificial Sequence Synthetic primer
cctctgtgatgctccttcacatta

SEQ ID NO: 32 - DNA, 20 bp, Artificial Sequence
  Description of Artificial Sequence Synthetic primer
ccgagtttacacgtcccctc

SEQ ID NO: 33 - DNA, 25 bp, Artificial Sequence
  Description of Artificial Sequence Synthetic primer
ggtctttgcttttatggctccagct

SEQ ID NO: 34 - DNA, 25 bp, Artificial Sequence
  Description of Artificial Sequence Synthetic primer
gaaacaaaactgtgtgctatgggga

SEQ ID NO: 35 - DNA, 25 bp, Artificial Sequence
  Description of Artificial Sequence Synthetic primer
tctcggcataagaaaagggtgaaga

SEQ ID NO: 36 - DNA, 24 bp, Artificial Sequence
  Description of Artificial Sequence Synthetic primer
gctccccttccgacagctatatag

SEQ ID NO: 37 - DNA, 25 bp, Artificial Sequence
  Description of Artificial Sequence Synthetic primer
tgctctgctggtgtaaaggattctg

SEQ ID NO: 38 - DNA, 21 bp, Artificial Sequence
  Description of Artificial Sequence Synthetic primer
tggagtaaggaaccggaccaa

SEQ ID NO: 39 - DNA, 24 bp, Artificial Sequence
  Description of Artificial Sequence Synthetic primer
tcacatcaagtattagtgcacgca

SEQ ID NO: 40 - DNA, 24 bp, Artificial Sequence
  Description of Artificial Sequence Synthetic primer
caacctgaccctgtccaatgaagg

SEQ ID NO: 41 - DNA, 21 bp, Artificial Sequence
  Description of Artificial Sequence Synthetic primer
cccggcattacctcactgaga

SEQ ID NO: 42 - DNA, 25 bp, Artificial Sequence
  Description of Artificial Sequence Synthetic primer
ggatcccgttagctactgtaggtgt

SEQ ID NO: 43 - DNA, 20 bp, Artificial Sequence
  Description of Artificial Sequence Synthetic primer
aggacaagcagtagcgctgc

SEQ ID NO: 44 - DNA, 25 bp, Artificial Sequence
  Description of Artificial Sequence Synthetic primer
aagaaaaaagcattcctggaaaggc

SEQ ID NO: 45 - DNA, 25 bp, Artificial Sequence
  Description of Artificial Sequence Synthetic primer
cagctatctcatttctgatgccttc

SEQ ID NO: 46 - DNA, 22 bp, Artificial Sequence
  Description of Artificial Sequence Synthetic primer
tgccaattgaaacttgctagca

SEQ ID NO: 47 - DNA, 25 bp, Artificial Sequence
  Description of Artificial Sequence Synthetic primer
cagctatctcatttctgatgccttc

SEQ ID NO: 48 - DNA, 43 bp, Gallus gallus
gctcccagttcaaatcttagaagcaagcacctgcacaaccaac

SEQ ID NO: 49 - DNA, 43 bp, Gallus gallus
gctcccagttcaaatctcagaagcaagcacctgcacaaccaac

SEQ ID NO: 50 - DNA, 43 bp, Gallus gallus
gttcccagttcaaatcttagaagcaagcacctgcacaaccaac

SEQ ID NO: 51 - DNA, 43 bp, Artificial Sequence; identical to SEQ ID NO: 50
  Description of Artificial Sequence Synthetic consensus sequence

SEQ ID NO: 52 - DNA, 50 bp, Gallus gallus
tgcaaacgggcccatttatgacagcacctgggaactccactggtggacac

SEQ ID NO: 53 - DNA, 50 bp, Gallus gallus
aaatcagttcccagttcaaatcttagaagcaagcacctgcacaaccaaga

SEQ ID NO: 54 - DNA, 50 bp, Gallus gallus
tcctgatacactactttttcccctaggaccgaaaagattgctcttaaagg

SEQ ID NO: 55 - DNA, 50 bp, Gallus gallus
aaatcagctcccagttcaaatcttagaagcaagcacctgcacaaccaaga

SEQ ID NO: 56 - DNA, 33 bp, Gallus gallus
ggtggacacaaatcagttcccagttcaaatctt

SEQ ID NO: 57 - DNA, 33 bp, Gallus gallus
ggtggacacaaatcagctcccagttcaaatctt